(19) United States
(12) Patent Application Publication
(10) Pub. No.: US 2024/0197795 A1
(43) Pub. Date: Jun. 20, 2024

(54) ASSESSMENT AND TREATMENT OF DEPRESSION, ANXIETY, OR RELATED DISORDERS

(71) Applicants: Boadie DUNLOP, Atlanta, GA (US); Oliver FIEHN, Davis, CA (US); Christopher Robert BRYDGES, Davis, CA (US); DUKE UNIVERSITY, Durham, NC (US); EMOORY UNIVERSITY, Atlanta, GA (US); UNIVERSITY OF CALIFORNIA, DAVIS, Davis, CA (US)

(72) Inventors: Rima F. Kaddurah-Daouk, Durham, NC (US); Augustus John Rush, JR., Durham, NC (US); Boadie W. Dunlop, Atlanta, GA (US); Oliver Fiehn, Davis, CA (US); Christopher R. Brydges, Davis, CA (US); W. Edward Craighead, Atlanta, GA (US); Helen Mayberg, Atlanta, GA (US)

(21) Appl. No.: 18/006,940

(22) PCT Filed: Jul. 27, 2021
(86) PCT No.: PCT/US2021/043287

§ 371 Date: Jan. 26, 2023

Related U.S. Application Data
(60) Provisional application No. 63/056,745, filed on Jul. 27, 2020.
(60) Provisional application No. 63/094,651, filed on Oct. 21, 2020.

(51) Int. Cl. A61K 35/742; A61K 45/06; G01N 33/53

(52) U.S. Cl. CPC A61K 35/742; A61K 45/06; G01N 33/5308; G01N 2800/304; G01N 2800/52

(57) ABSTRACT

Described herein are methods of identifying, characterizing, and treating depression and anxiety in a subject by modulating indoles associated with tryptophan metabolism in the subject and subject's gut microbiome.

### CROSS-REFERENCE TO RELATED APPLICATIONS

This application claims priority to U.S. Provisional Patent Application Nos. 63/056,745, filed on Jul. 27, 2020 and 63/094,651 filed on Oct. 21, 2020, each of which is incorporated by reference herein in its entirety.

### FEDERALLY SPONSORED RESEARCH

This invention was made with government support under grant number R01MH108348 awarded by the National Institutes of Health. The government has certain rights in the invention.

### TECHNICAL FIELD

Described herein are methods of identifying, characterizing, and treating depression and anxiety in a subject by modulating indoles associated with tryptophan metabolism in the subject and subject's gut microbiome.

### BACKGROUND

Tryptophan metabolism can lead to production of serotonin, a metabolite that is implicated in depression discoveries that have led to the development of selective serotonin reuptake inhibitors (SSRIs), a class of antidepressant drugs that increase serotonin signaling in the central nervous system by blocking its presynaptic reuptake. Representatives of SSRIs include escitalopram, citalopram, and sertraline. In addition, tryptophan metabolism leads to the production of kynurenine, melatonin, related methoxyindoles, and a series of indoles that are produced by human and gut bacteria co-metabolism. Some of these indoles include indole-3-acetate (IAA), indole-3-lactate (ILA), indole-3-propionic acid (13PA), and indoxyl sulfate (IS), among other indoles. Indoxyl sulfate is a uremic toxin derived from indole by further metabolism in the liver. These indoles are known to be regulators of aryl hydrocarbon receptor and/or nuclear receptors such as PXR which are implicated in energy hemostasis and in immune function regulation. However, it is unknown whether indoles are associated with symptoms of depression and anxiety.

What is needed are methods for methods of identifying, characterizing, and treating depression and anxiety based on indoles associated with tryptophan metabolism in the subject and subject's gut microbiome. Increasing evidence suggests that the gut microbiome produces chemicals some might be toxic and can impact behavior and mood. Drugs that target gut microbiome products can emerge as new therapies for the treatment of neuropsychiatric diseases.

### SUMMARY

Described herein is the elucidation of the roles of indoles produced by bacteria in the human gut which can be modulated to affect anxiety and depression.

One embodiment described herein is a method for treating a subject suffering from, or at risk of suffering from, depression, major depressive disorder, anxiety, or a combination thereof by modulating the gut microbiome composition or enzymatic activity of the subject, the method comprising: (a) increasing the level of indole-3-propionic acid producing bacteria; (b) decreasing the level of indoxyl sulfate producing bacteria; (c) inhibiting bacterial enzymes that produce indoxyl sulfate; (d) activating bacterial enzymes that produce indole-3-propionic acid; or a combination of any of (a)-(d). In one aspect, the subject is undergoing cognitive behavioral therapy, treatment with an anti-depressant, or a combination thereof. In another aspect, the anti-depressant comprises one or more of selective serotonin reuptake inhibitors (SSRI), tricyclic anti-depressants (TCA), selective serotonin and norepinephrine reuptake inhibitors (SNRI), monoamine oxidase inhibitors (MAOI), anxiolytics, antipsychotics, or combinations thereof. In another aspect, the anti-depressant comprises one or more of citalopram (Celexa®), escitalopram (Lexapro®), duloxetine (Cymbalta®), fluoxetine (Prozac®), paroxetine (Paxil®), sertraline (Zoloft®), trazodone (Desyrel®), lorazepam (Ativan®), oxazepam (Serax®), aripiprazole (Abilify®), cozapine (Clozaril®), haloperidol (Haldol®), olanzapine (Zyprexa®), quetiapine (Seroquel®), risperidone (Risperdal®), ziprasidone (Geodon®), amitriptyline, amoxapine, desipramine (Norpramin®), doxepin, imipramine (Tofranil®), nortriptyline (Pamelor®), protriptyline, trimipramine, or combinations thereof. In another aspect, the anti-depressant comprises escitalopram (Lexapro®), duloxetine (Cymbalta®), or a combination thereof. In another aspect, the indole-3-propionic acid producing bacteria comprise Clostridium sporogenes. In another aspect, increasing the level of indole-3-propionic acid producing bacteria comprises obtaining and administering to the subject probiotic Clostridium sporogenes. In another aspect, the indoxyl sulfate producing bacteria comprise tryptophanase expressing bacteria. In another aspect, the method further comprises administering to the subject one or more therapeutic agents to trap and sequester indoxyl sulfate. In another aspect, the therapeutic agent comprises AB-2004 or activated charcoal. In another aspect, the method further comprises administering to the subject one or more therapeutic agents to inhibit enzymatic activity of sulfotransferase liver enzymes that produce indoxyl sulfate. In another aspect, the method prevents indoxyl sulfate from crossing the blood-brain barrier. In another aspect, the method further comprises administering to the subject one or more therapeutic agents to inhibit aryl hydrocarbon receptor activity of intestinal immune cells. In another aspect, the method further comprises: measuring a concentration of one or more of the subject's tryptophan gut metabolites comprising one or more of indoxyl sulfate, indole-3-propionic acid, 5-methoxytryptamine, indole-3-acetate, indole-3-lactate, or combinations thereof; and determining ratios of indoxyl sulfate concentration to the concentrations of indole-3-propionic acid, 5-methoxytryptamine, indole-3-acetate, or indole-3-lactate. In another aspect, the measuring the concentration of one or more tryptophan gut metabolites is repeated at least twice; wherein a first measurement is taken as a baseline reading and a second or subsequent measurement is taken following modulation of the gut microbiome composition, enzymatic activity, or combination thereof of the subject. In another aspect, the measured concentrations and determined ratios are compared to a healthy control. In another aspect, ratios are correlated with Hamilton Anxiety scores, Hamilton Depression scores, or Quick Inventory of Depressive Symptoms. In another aspect, the method modulates a concentration of one or more tryptophan gut metabolites in the subject selected from indoxyl sulfate, indole-3-propionic acid, 5-methoxytryptamine, indole-3-acetate, indole-3-lactate, or combinations thereof. In another aspect, the method reduces the concentration of indoxyl sulfate in the subject. In another aspect, the method increases the concentration of indole-3-propionic acid in the subject. In another aspect, the method further comprises assessing disease severity of the subject prior to, during, or following modulation of the gut microbiome composition, enzymatic activity, or combination thereof of the subject. In another aspect, assessing disease severity comprises the use of one or more disease assessment tests selected from a Quick Inventory of Depressive Symptomatology Self-Report (QIDS-SR), a Hamilton Anxiety Rating Scale (HAM-A), or a Hamilton Depression Rating Scale (HAM-D).

Another embodiment described herein is a method for treating a subject suffering from, or at risk of suffering from, depression, major depressive disorder, anxiety, or combination thereof by characterizing the gut microbiome metabolic profile of the subject, the method comprising: (a) obtaining one or more biological samples from the subject; (b) measuring a baseline concentration of one or more tryptophan gut metabolites selected from indoxyl sulfate, indole-3-propionic acid, 5-methoxytryptamine, indole-3-acetate, indole-3-lactate, or combinations thereof; (c) determining baseline ratios of indoxyl sulfate concentration to the concentrations of indole-3-propionic acid, 5-methoxytryptamine, indole-3-acetate, and indole-3-lactate, wherein increased baseline ratios of indoxyl sulfate indicate that the subject is suffering from, or at risk of suffering from, depression, major depressive disorder, anxiety or combination thereof; and (d) administering a therapy to the subject when increased ratios of indoxyl sulfate are determined. In one aspect, the subject is undergoing cognitive behavioral therapy, treatment with an anti-depressant, or a combination thereof. In another aspect, the anti-depressant comprises one or more of selective serotonin reuptake inhibitors (SSRI), tricyclic anti-depressants (TCA), selective serotonin and norepinephrine reuptake inhibitors (SNRI), monoamine oxidase inhibitors (MAOI), anxiolytics, antipsychotics, or combinations thereof. In another aspect, the anti-depressant comprises one or more of citalopram (Celexa®), escitalopram (Lexapro®), duloxetine (Cymbalta®), fluoxetine (Prozac®), paroxetine (Paxil®), sertraline (Zoloft®), trazodone (Desyrel®), lorazepam (Ativan®), oxazepam (Serax®), aripiprazole (Abilify®), clozapine (Clozaril®), haloperidol (Haldol®), olanzapine (Zyprexa®), quetiapine (Seroquel®), risperidone (Risperdal®), ziprasidone (Geodon®), amitriptyline, amoxapine, desipramine (Norpramin®), doxepin, imipramine (Tofranil®), nortriptyline (Pamelor®), protriptyline, trimipramine, or combinations thereof. In another aspect, the anti-depressant comprises escitalopram (Lexapro®), duloxetine (Cymbalta®), or a combination thereof. In another aspect, the measuring the concentration of one or more tryptophan gut metabolites is repeated at least twice, wherein a first measurement is taken as a baseline reading and a second or subsequent measurement is taken following administration of the therapy to the subject. In another aspect, the measured baseline concentrations and determined baseline ratios are compared to a healthy control. In another aspect, the therapy comprises one or more therapeutic agents to trap and sequester indoxyl sulfate. In another aspect, the therapeutic agent comprises AB-2004 or activated charcoal. In another aspect, the therapy comprises a probiotic gut bacterial strain that produces indole-3-propionic acid and does not produce indoxyl sulfate. In another aspect, the probiotic gut bacterial strain comprises Clostridium sporogenes. In another aspect, the therapy reduces tryptophanase expressing bacteria. In another aspect, the therapy comprises one or more therapeutic agents to inhibit enzymatic activity of sulfotransferase liver enzymes that produce indoxyl sulfate. In another aspect, the therapy comprises one or more therapeutic agents to inhibit aryl hydrocarbon receptor activity of intestinal immune cells. In another aspect, the method further comprises performing functional magnetic resonance imaging of the subject prior to, during, or following administration of the therapy to the subject to monitor changes in brain resting state functional connectivity. In another aspect, the method further comprises assessing disease severity of the subject prior to, during, and following administration of the therapy to the subject. In another aspect, assessing disease severity comprises the use of one or more disease assessment tests selected from a Quick Inventory of Depressive Symptomatology Self-Report (QIDS-SR), a Hamilton Anxiety Rating Scale (HAM-A), or a Hamilton Depression Rating Scale (HAM-D).

Another embodiment described herein is a method for stratifying one or more subjects suffering from a neuropsychiatric disease, disorder, or condition, including depression, major depressive disorder, anxiety, or combinations thereof into subgroups based on individual gut microbiome composition, enzymatic activity, concentrations and ratios of metabolites produced by gut bacteria, or combinations thereof, the method comprising: (a) obtaining one or more biological samples from the subjects; (b) measuring a baseline concentration of one or more tryptophan gut metabolites selected from indoxyl sulfate, indole-3-propionic acid, 5-methoxytryptamine, indole-3-acetate, indole-3-lactate, or combinations thereof; (c) determining a baseline ratio of indoxyl sulfate concentration to concentrations of indole-3-propionic acid, 5-methoxytryptamine, indole-3-acetate, indole-3-lactate, or combinations thereof; (d) stratifying the subjects into subgroups based on the determined baseline ratios of indoxyl sulfate concentration to concentrations of indole-3-propionic acid, 5-methoxytryptamine, indole-3-acetate, indole-3-lactate, or combinations thereof; and (e) assessing disease severity of subjects in each stratified subgroup. In one aspect, increased ratios of indoxyl sulfate concentration to concentrations of indole-3-propionic acid, 5-methoxytryptamine, indole-3-acetate, and indole-3-lactate indicate that the subject is suffering from, or at risk of suffering from, depression, major depressive disorder, anxiety, or combinations thereof. In another aspect, the measured baseline concentrations and determined baseline ratios are compared to a healthy control. In another aspect, assessing disease severity of subjects in each stratified subgroup comprises using one or more disease assessment tests selected from a Quick Inventory of Depressive Symptomatology Self-Report (QIDS-SR), a Hamilton Anxiety Rating Scale (HAM-A), or a Hamilton Depression Rating Scale (HAM-D). In another aspect, the method further comprises administering a therapy to subjects in each stratified subgroup based on the assessed disease severity and the determined baseline ratios of indoxyl sulfate concentration to concentrations of indole-3-propionic acid, 5-methoxytryptamine, indole-3-acetate, indole-3-lactate, or combinations thereof. In another aspect, the therapy comprises modulating the gut microbiome composition or enzymatic activity of the subject by: (a) increasing the level of indole-3-propionic acid producing bacteria; (b) decreasing the level of indoxyl sulfate producing bacteria; (c) inhibiting bacterial enzymes that produce indoxyl sulfate; (d) activating bacterial enzymes that produce indole-3-propionic acid; or a combination of any of (a)-(d).

Another embodiment described herein is the use of a therapeutic agent to trap and sequester indoxyl sulfate for the treatment of a subject suffering from, or at risk of suffering from, depression, major depressive disorder, anxiety, or combinations thereof.

Another embodiment described herein is the use of an indole-3-propionic acid producing probiotic gut bacterial strain for the treatment of a subject suffering from, or at risk of suffering from, depression, major depressive disorder, anxiety, or combinations thereof. In one aspect, the probiotic gut bacterial strain comprises Clostridium sporogenes.

Another embodiment described herein is the use of a therapeutic agent to inhibit enzymatic activity of sulfotransferase liver enzymes that produce indoxyl sulfate for the treatment of a subject suffering from, or at risk of suffering from, depression, major depressive disorder, anxiety, or combinations thereof.

Another embodiment described herein is the use of a therapeutic agent to inhibit aryl hydrocarbon receptor activity of intestinal immune cells for the treatment of a subject suffering from, or at risk of suffering from, depression, major depressive disorder, anxiety, or combinations thereof.

Another embodiment described herein is a method for treating a subject suffering from, or at risk of suffering from, immune metabolic depression, anxiety, or combinations thereof by characterizing the gut microbiome metabolic profile of the subject, the method comprising: (a) obtaining one or more biological samples from the subject; (b) measuring a concentration of one or more tryptophan gut metabolites selected from indoxyl sulfate, indole-3-propionic acid, 5-methoxytryptamine, indole-3-acetate, indole-3-lactate, or combinations thereof; (c) determining ratios of indoxyl sulfate concentration to the concentrations of indole-3-propionic acid, 5-methoxytryptamine, indole-3-acetate, indole-3-lactate, or combinations thereof, wherein increased ratios of indoxyl sulfate indicate that the subject is suffering from, or at risk of suffering from, depression, major depressive disorder, anxiety, or combinations thereof; (d) measuring aryl hydrocarbon receptor activity of intestinal immune cells; and (e) administering a therapy to the subject when increased ratios of indoxyl sulfate are determined. In one aspect, the measured concentrations and determined ratios are compared to a healthy control. In another aspect, the therapy comprises modulating the gut microbiome composition or enzymatic activity of the subject by: (a) increasing the level of indole-3-propionic acid producing bacteria; (b) decreasing the level of indoxyl sulfate producing bacteria; (c) inhibiting bacterial enzymes that produce indoxyl sulfate; (d) activating bacterial enzymes that produce indole-3-propionic acid; or a combination of any of (a)-(d). In another aspect, the therapy comprises one or more therapeutic agents to inhibit aryl hydrocarbon receptor activity of intestinal immune cells.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a diagram of tryptophan human gut bacterial co-metabolism leading to production of indoles including IPA, IAA, ILA, and IS. Abbreviations: 3-HAA: 3-Hydroxyanthranilic Acid; 3H-KYN: 3-Hyroxykynurenine; 5-HTP: 5-hydroxytryptophan; AAAD: Aromatic Amino Acid Decarboxylase; AANAT: Aralkylamine N-Acetyltransferase; acdA: acyl-CoA dehydrogenase; AraT: Aromatic Amino Acid Aminotransferase: ASMT: Acetylserotonin O-Methyltransferase; fldBC: phenyllactate dehydratase; fldH: phenyllactate dehydrogenase; IA: Indole Acrylic acid; IAA: Indole Acetic Acid; IAAld, indole-3-Acetaldehyde; IAld: Indole-3-Aldehyde; IAM: indole-3-Acetamide; IDO: Indolamine 2,3-Dioxygenase; ILA: Indole-3-LacticAcid; IPA: Indole-3-Propionic Acid; IPYA: Indole-3-Pyruvate; KAT: Kynurenine aminotransferase; KMO: Kynurenine 3-Monooxygenase; KYNU: Kynureninase; MAO: Monoamine Oxidase; NAD: Nicotinamide Adenine Dinucleotide; porB, C: pyruvate: ferredoxin oxidoreductase B and C; TDO: Tryptophan 2,3-Dioxygenase; TMO: Tryptophan 2-Monooxygenase; TNA: Tryptophanase; TpH: Tryptophan Hydroxylase; TrD: Tryptophan Decarboxylase.

FIG. 2 shows a heat map of Spearman rank correlations between baseline indole intensity/ratio and baseline Hamilton Rating Scale for Depression (HRSD) score for male, female, and all study participants. A positive correlation (shown in red) indicates more severe depression associated with greater abundance of metabolite.

FIG. 3 shows a heat map of Spearman rank correlations between baseline Quick Inventory of Depressive Symptomatology-Self-Rated (QIDS-SR) items and baseline indole intensity/ratio for female male, and all study participants. The 16 QIDS-SR items include: QIDS1—Falling asleep; QIDS2—Sleep during the night; QIDS3—Waking up too early; QIDS4—Sleeping too much; QIDS5—Feeling sad; QIDS6—Decreased appetite; QIDS7—Increased appetite; QIDS8 Decreased weight; QIDS9—Increased weight; QIDS10—Concentration/decision making; QIDS11—View of myself; QIDS12—Thoughts of death or suicide; QIDS13—General interest; QIDS14—Energy level; QIDS15—Feeling slowed down; and QIDS16—Feeling restless. A positive correlation (shown in red) indicates more severe depression phenotype associated with greater abundance of metabolite.

FIG. 4 shows a heat map of Mann-Whitney U between-group comparisons between baseline comorbidities and baseline indole intensity/ratio for female, male, and all study participants. Anxious=diagnosed with anxious comorbid disorder vs. not diagnosed. High/low anxiety=binary grouping based on cut-off score of 15 on Hamilton Rating Scale for Anxiety (HRSA). Depression severity=binary grouping based on cut-off score of 20 on Hamilton Rating Scale for Depression (HRSD). Fold change>1 (shown in red) indicates greater abundance of metabolite in more extreme group.

FIG. 5 shows a heat map of Spearman rank correlations between 12-week change in indole intensity/ratio and treatment groups for female, male, and all study participants. Fold change>1 (shown in red) indicates increases in metabolite from baseline to week 12.

FIG. 6 shows a heat map of Spearman rank correlations between 12-week change in indole intensity/ratio and 12-week change in depression for female, male, and all study participants with different antidepressant treatments. Positive correlation (shown in red) indicates improvement of depression associated with decrease of metabolite.

FIG. 7A shows a heat map of Spearman rank correlations between 12-week change in indole intensity/ratio and 12-week change in depression phenotype (QIDS-SR items 1-16) for all study participants with different antidepressant treatments. Positive correlation (shown in red) indicates improvement of depression phenotype associated with increase of metabolite.

FIG. 7B shows a heat map of Spearman rank correlations between 12-week change in indole intensity/ratio and 12-week change in depression phenotype (QIDS-SR items 1-16) for female study participants with different antidepressant treatments. Positive correlation (shown in red) indicates improvement of depression phenotype associated with increase of metabolite.

FIG. 7C shows a heat map of Spearman rank correlations between 12-week change in indole intensity/ratio and 12-week change in depression phenotype (QIDS-SR items 1-16) for male study participants with different antidepressant treatments. Positive correlation (shown in red) indicates improvement of depression phenotype associated with increase of metabolite.

FIG. 8 shows a heat map of Spearman rank correlations between baseline indole intensity/ratio and participant demographic variables.

FIG. 9 shows a heat map of partial Spearman rank correlations between baseline indole abundance/ratio and Hamilton Anxiety scores and Hamilton Depression scores, after accounting forage, sex, and BMI.

FIG. 10 shows a heat map of partial Spearman rank correlations between baseline indole abundance/ratio and QIDS-SR items and total score, after accounting for age, sex, and BMI. Abbreviations: QIDS-SR: 16-item Quick Inventory of Depressive Symptomatology-Self-Rated.

FIG. 11A-C show images of resting state functional connectivity of subcallosal cingulate cortex (SCC) associations with peripheral indoxyl sulfate abundances and psychic anxiety scores. FIG. 11A shows SCC functionally connected regions indicating a significant correlation with indoxyl sulfate abundances. Green circles identify regions incorporated into the mediation models. FIG. 11B shows SCC functionally connected regions indicating a significant correlation with psychic anxiety scores. The orange circle identifies the right premotor region. FIG. 11C shows conjunction analysis: SCC functionally connected region indicating a significant correlation with both indoxyl sulfate abundances and psychic anxiety scores. The red circles indicate the only region to emerge in this analysis, which is the right premotor region. Abbreviations: HAMPSY: Psychic anxiety subscore of the Hamilton Anxiety Rating Scale. SCC-FC: subcallosal cingulate cortex functional connectivity.

FIG. 12A-D show that the impact of indoxyl sulfate on psychic anxiety scores is mediated by its effects on the resting state functional connectivity between the subcallosal cingulate cortex and the right premotor region. FIG. 12A shows the association between indoxyl sulfate and psychic anxiety scores. FIG. 12B shows a mediation model incorporating the overlapping area, right premotor region, indicating that the effect of indoxyl sulfate on psychic anxiety is mediated via its effects on the functional connectivity between the SCC and right premotor region. FIG. 12C shows significant SCC-FC correlations between the right anterior insula, right anterior midcingulate cortex, and right premotor region. FIG. 12D shows a full mediation model incorporating the three regions showing significant SCC-FC correlations with indoxyl sulfate abundances. Although indoxyl sulfate is significantly correlated with all three regions, only the pathway through the right premotor region significantly mediates the effect of indoxyl sulfate on psychic anxiety. Black lines indicate significant associations within the models; grey lines indicate insignificant associations. Red lines indicate significant mediation of indoxyl sulfate on psychic anxiety through the indirect pathway of right premotor SCC-FC. Abbreviations: HAMPSY: Psychic anxiety subscore of the Hamilton Anxiety Rating Scale. SCC-FC: subcallosal cingulate cortex functional connectivity.

FIG. 13A-D show images and graphs of the correlation between peripheral indoxyl sulfate intensities and resting state functional connectivity (RSFC) of subcallosal cingulate cortex (SCC). FIG. 13A shows the RSFC of right anterior insula and indicates that the SCC was significantly correlated with serum indoxyl sulfate intensities, corrected at p<0.001. FIG. 13B shows a scatterplot and trendline of correlation from FIG. 13A (R²=0.241). FIG. 13C shows that the correlation of indoxyl sulfate was significant with SCC-anterior insula RSFC bilaterally, using correction of p<0.005. FIG. 13D shows that indoxyl sulfate significantly correlated with SCC-right supplementary motor area RSFC.

### DETAILED DESCRIPTION

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the terms “effective amount” or “therapeutically effective amount,” refers to a substantially non-toxic, but sufficient amount of an agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder, or condition.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive index Model (AIM) methodology. Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, e.g., to determine the performance of each marker in identifying a patient having CRC. Alternatively, cutoff values may be determined by a quartile analysis of biological samples of a patient group. For example, a cutoff value may be determined by selecting a value that corresponds to any value in the 25^{th}-75^{th} percentile range, preferably a value that corresponds to the 25^{th} percentile, the 50^{th} percentile or the 75^{th} percentile, and more preferably the 75^{th} percentile. Such statistical analyses may be performed using any method known in the art and can be implemented through any number of commercially available software packages. The healthy or normal levels or ranges for a target or for a protein activity may be defined in accordance with standard practice.

As used herein, “healthy control” or “normal control” refers to a human subject that is not suffering from, or at risk of suffering from, a neuropsychiatric disease or disorder including, but not limited to, depression, anxiety, major depressive disorder, or combinations thereof.

“Sample” or “test sample” as used herein can mean any sample in which the presence and/or level of a target is to be detected or determined or any sample comprising an agent or cell as described herein. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

As used herein, “depression” refers to a mood disorder that causes a persistent feeling of sadness and loss of interest. As used herein, “depression” includes subclinical characteristics associated with depression such as sadness, loss of interest in activities, loss of appetite, anhedonia, insomnia, changes in sleep, difficulty falling asleep, waking during the night, restless sleep, waking too early, sleeping too much, low energy level, lack of concentration, diminished or altered daily behavior, low self-esteem, suicidal thoughts, anxiety coupled with depression, or combinations thereof. As used herein “major depressive disorder” refers to a mental health disorder characterized by persistent feeling of sadness or loss of interest that characterizes major depression can lead to a range of behavioral and physical symptoms causing significant impairment in daily life. These may include depressed mood, loss of interest in activities, changes in sleep, appetite, energy level, concentration, daily behavior, self-esteem, or suicide ideation.

As used herein, “anxiety” refers to anxiety disorders including generalized anxiety disorder, panic attacks, obsessive-compulsive disorders, phobias, and post-traumatic stress disorders. Symptoms include feelings of apprehension or dread or impending doom, feeling tense or jumpy, restlessness or irritability, difficulty controlling feelings of worry, anticipating the worst and being watchful for signs of danger, difficulty concentrating or mind going blank, and physical symptoms including heart palpitations, pounding or racing heart, shortness of breath, sweating, tremors or shaking, muscle tension, headaches, fatigue, insomnia, upset stomach, frequent urination, or diarrhea.

The gut microbiota impacts numerous aspects of human health and disease, including neuropsychiatric disorders. The “microbiota-gut-brain axis” refers to a bidirectional communication pathway that connects the central nervous system (CNS), the gut, and the microbial community that inhabits the gastrointestinal tract. Within this axis, the gut microbiota modulates central processes through the activation of neuronal pathways (e.g., the vagus nerve) as well as through the production of microbial metabolites and immune mediators that can trigger changes in neurotransmission, neuroinflammation, and behavior.

Disruptions to the gut microbiome have been correlated with several neurological disorders, including Parkinson's disease, autism spectrum disorder, schizophrenia, and major depressive disorder (MDD), though the specific mechanisms that underlie the role of the gut microbiota in these diseases is not fully understood. However, research in preclinical rodent models shows that the gut microbiota is sufficient to after host behavior, as shown by the increase in anxiety- and depressive-like behaviors in rodents after fecal microbiota transfer from humans with depression relative to those that received transfer of fecal microbiota from demographic controls. Further, transferred microbes resulted in altered metabolic states in the recipient mice that displayed depressive-like symptoms. These data implicate the gut microbiota as direct contributors to behaviors associated with depression and anxiety through their metabolic effects. In this study, gut microbiota-associated tryptophan metabolism was explored, and the levels of metabolites are correlated to clinical symptoms and severity of depression and anxiety in humans.

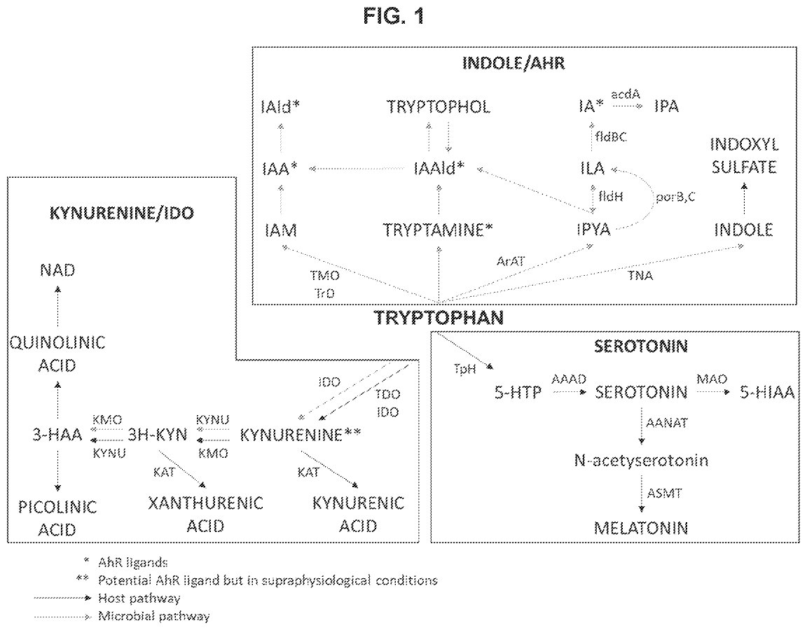

Tryptophan is an essential amino acid that can be metabolized in the gastrointestinal tract via the serotonin, kynurenine, and indole metabolic pathways (FIG. 1), which have been associated with human maladies including autoimmunity, inflammatory diseases, metabolic syndrome, and neurological diseases including depression and anxiety disorders. Strikingly, the gut microbiota is exclusively responsible for the conversion of tryptophan in the indole pathway, as there are no detectable levels of indole or indole derivatives in gnotobiotic mice that lack a gut microbiome. Analysis of biosynthetic pathways found that the genes necessary to make indole and indole derivatives, such as indole-3-propionic acid (IPA), indole-3-acetic acid (IAA), and indole-3-lactic acid (ILA), are found exclusively in the gut microbiome but not in mammalian genomes (FIG. 1). These indoles can have important immunomodulatory effects and are potent agonists for aryl hydrocarbon receptors (AHRs), which regulate host immunity and barrier function at mucosal sites.

Indole derivatives can also affect immune status in the brain, as some indole derivatives (e.g., IPA and IAA) have anti-inflammatory effects on neurodegenerative diseases in the experimental autoimmune encephalomyelitis (EAE) mouse model of multiple sclerosis as well as in a cell line model of Alzheimer's disease. Other indole derivatives can be further metabolized by host processes into molecules that may be harmful to human health. Specifically, indole can be sulfonated in the liver into uremic toxin indoxyl sulfate (IS), which crosses the blood-brain barrier (FIG. 1). IS, which is normally cleared via the kidneys and excreted in the urine, is associated with cardiovascular disease in patients who have chronic kidney disease via induction of oxidative stress in endothelial cells, and peripheral IS concentrations are associated with diminished cognitive function in renal dialysis patients.

IS also associated with both neurodevelopmental and neurodegenerative diseases, as levels of IS are increased in patients who have an autism spectrum disorder or Parkinson's disease. Although the mechanistic role of IS in these diseases is unknown, IS increases levels of oxidative stress and pro-inflammatory cytokine signaling in astrocytes and mixed glial cells during in vitro administration, which suggests that inflammation and reactive oxygen species may be involved. Further, IS has been associated with behavioral defects in preclinical models of anxiety and depression. The administration of IS into rodents' drinking water results in increased concentrations of IS in the brain and increased blood-brain barrier permeability in an AHR-dependent manner, with accompanying increases in anxiety and cognitive deficits. Monocolonization experiments with indole-producing Escherichia coli and isogenic mutants have shown that indole production by gut bacteria is sufficient to drive increases in anxiety- and depressive-like behavior in rats.

Collectively, preclinical and clinical data indicate that indole derivatives provide excellent models for studying the microbiota-gut-brain axis given their connection to central immune regulators (i.e., AHR), their link to human neurological diseases, and the exclusivity of indole production to gut microbes.

To date, the effects of peripheral metabolic concentrations on neural functioning have received little study, likely due to the paucity of datasets that contain concurrently collected metabolomic and neuroimaging measures. Such research is crucial for determining how changes in peripheral systems may yield alterations in brain function that can produce clinically relevant symptoms such as depression, anxiety, or cognitive impairment.

Using blood samples stored from the Prediction of Remission in Depression to Individual and Combined Treatments (PReDICT) study, which was a large study of treatment-naïve patients with MDD, the levels of four indole derivatives (IPA, IAA, ILA, IS) were measured to investigate whether the levels and ratios of indoles at baseline prior to treatment correlate with depression and anxiety severity at baseline, and specific individual symptoms of depression. This study also investigated whether changes in symptoms after treatment with duloxetine, escitalopram, or cognitive behavioral therapy (CBT) can be predicted by baseline levels of indoles, and whether symptom changes correlate with changes in levels of indoles after treatment. Furthermore, functional Magnetic Resonance Imaging (fMRI) was used to determine whether relationships exist between baseline peripheral metabolic concentrations of indoles and brain resting state functional connectivity.

Also described is a randomized-controlled trial (termed PReDICT) performed by the inventors that aimed to identify moderators and mediators of improvement with ADMs (escitalopram or duloxetine) or CBT (behavioral therapy). In the study, approximately 300 blood samples were collected using GCTOF platform part of West Coast Metabolomics Center (UC Davis). Samples were evaluated to determine the following: (a) metabolites at baseline correlated with disease severity (QIDS or HAMD scores) and with response to treatment; (b) change in levels of metabolites correlated with change in disease severity; and (c) if baseline level of metabolites or change in metabolite levels correlate with sub clinical phenotypes or symptoms in depression including perturbed sleep, fatigue, anxiety, depression appetite among others.

The results of the study showed: (a) Higher levels of indoxyl sulfate at baseline associated with higher levels of depression at baseline HRSD (prior to treatment); (b) Higher levels of indole-3-propionic acid at baseline associated with lower levels of depression at baseline HRSD (prior to treatment); (c) Higher levels of indoxyl sulfate at baseline associated with more severe symptoms of sub clinical phenotypes including sleeping too much; feeling sad; decreased appetite; low general interest; feeling restless; (d) 5-methoxytryptamine decreased significantly as a result of medication parallels decrease in serotonin; (e) Escitalopram and duloxetine had modest effects on levels of indoxyl sulfate suggesting that other types of drugs might be needed to ameliorate its negative effects in depressed patients; (f) Decreases from baseline to week 12 of indoxyl sulfate associated with alleviated ‘falling asleep’, thoughts of death or suicide, and feeling slowed down (QIDS 1, 12, and 15) in escitalopram group; (g) Increases from baseline to week 12 of tryptophan associated with alleviated ‘sleep during the night’ (QIDS 2); and (8) Decreases in Indole-3-acetate, indole-3-lactate, indoxyl sulfate, and tryptophan all associated with improved energy level (QIDS 14).

There are numerous therapies that can be administered to a subject in step (c) based on the results of the methods provided herein. For example, drugs being developed for trapping indoles including indoxyl sulfate in other CNS diseases like autism will be useful for treatment of depression. Examples of such products include AB-2004 and a large number of related compounds. Activated charcoal might be useful. Accordingly, another aspect of the present disclosure provides stratifying depressed patients based on their metabolic profiles as provided herein and based on the results, define who might benefit from use of such medications. In another embodiment, resulting treatments for the methods provided herein may further comprise obtaining and administering to the subject bacterial strains found in the gut and/or performing metabolic reconstruction in order to regulate different indoles found in the subject's gut to thereby treat the depression. In other embodiments, therapies include enriching for bacterial strains that produce IPA instead of indoxyl sulfate or blocking tryptophanase bacteria and its products indoxyl sulfate. Also blocking liver enzymes that lead to sulfation of indole to indoxyl sulfate will be helpful for depressed patients. In other embodiments, utilizing a combination of antidepressants and/or drugs that target the gut microbiome are used to better control symptoms in some of patients with major depression, including helping them sleep better and have higher energy levels.

In yet another embodiment, the present disclosure provides methods of regulating nuclear receptors and aryl hydrocarbon receptors for developing novel classed of antidepressants for treating symptoms of disease and subclinical phenotypes.

One embodiment described herein is a method for treating a subject suffering from, or at risk of suffering from, depression, major depressive disorder, anxiety, or a combination thereof by modulating the gut microbiome composition or enzymatic activity of the subject, the method comprising: (a) increasing the level of indole-3-propionic acid producing bacteria; (b) decreasing the level of indoxyl sulfate producing bacteria; (c) inhibiting bacterial enzymes that produce indoxyl sulfate; (d) activating bacterial enzymes that produce indole-3-propionic acid; or a combination of any of (a)-(d). In one aspect, the subject is undergoing cognitive behavioral therapy, treatment with an anti-depressant, or a combination thereof. In another aspect, the anti-depressant comprises one or more of selective serotonin reuptake inhibitors (SSRI), tricyclic anti-depressants (TCA), selective serotonin and norepinephrine reuptake inhibitors (SNRI), monoamine oxidase inhibitors (MAOI), anxiolytics, antipsychotics, or combinations thereof. In another aspect, the anti-depressant comprises one or more of citalopram (Celexa®), escitalopram (Lexapro®), duloxetine (Cymbalta®), fluoxetine (Prozac®), paroxetine (Paxil®), sertraline (Zoloft®), trazodone (Desyrel®), lorazepam (Ativan®), oxazepam (Serax®), aripiprazole (Abilify®), clozapine (Clozaril®), haloperidol (Haldol®), olanzapine (Zyprexa®), quetiapine (Seroquel®), risperidone (Risperdal®), ziprasidone (Geodon®), amitriptyline, amoxapine, desipramine (Norpramin®), doxepin, imipramine (Tofranil®), nortriptyline (Pamelor®), protriptyline, trimipramine, or combinations thereof. In another aspect, the anti-depressant comprises escitalopram (Lexapro®), duloxetine (Cymbalta®), or a combination thereof. In another aspect, the indole-3-propionic acid producing bacteria comprise Clostridium sporogenes. In another aspect, increasing the level of indole-3-propionic acid producing bacteria comprises obtaining and administering to the subject probiotic Clostridium sporogenes. In another aspect, the indoxyl sulfate producing bacteria comprise tryptophanase expressing bacteria. In another aspect, the method further comprises administering to the subject one or more therapeutic agents to trap and sequester indoxyl sulfate. In another aspect, the therapeutic agent comprises AB-2004 or activated charcoal. In another aspect, the method further comprises administering to the subject one or more therapeutic agents to inhibit enzymatic activity of sulfotransferase liver enzymes that produce indoxyl sulfate. In another aspect, the method prevents indoxyl sulfate from crossing the blood-brain barrier. In another aspect, the method further comprises administering to the subject one or more therapeutic agents to inhibit aryl hydrocarbon receptor activity of intestinal immune cells. In another aspect, the method further comprises: measuring a concentration of one or more of the subject's tryptophan gut metabolites comprising one or more of indoxyl sulfate, indole-3-propionic acid, 5-methoxytryptamine, indole-3-acetate, indole-3-lactate, or combinations thereof: and determining ratios of indoxyl sulfate concentration to the concentrations of indole-3-propionic acid, 5-methoxytryptamine, indole-3-acetate, or indole-3-lactate. In another aspect, the measuring the concentration of one or more tryptophan gut metabolites is repeated at least twice; wherein a first measurement is taken as a baseline reading and a second or subsequent measurement is taken following modulation of the gut microbiome composition, enzymatic activity, or combination thereof of the subject. In another aspect, the measured concentrations and determined ratios are compared to a healthy control. In another aspect, ratios are correlated with Hamilton Anxiety scores, Hamilton Depression scores, or Quick Inventory of Depressive Symptoms. In another aspect, the method modulates a concentration of one or more tryptophan gut metabolites in the subject selected from indoxyl sulfate, indole-3-propionic acid, 5-methoxytryptamine, indole-3-acetate, indole-3-lactate, or combinations thereof. In another aspect, the method reduces the concentration of indoxyl sulfate in the subject. In another aspect, the method increases the concentration of indole-3-propionic acid in the subject. In another aspect, the method further comprises assessing disease severity of the subject prior to, during, or following modulation of the gut microbiome composition, enzymatic activity, or combination thereof of the subject. In another aspect, assessing disease severity comprises the use of one or more disease assessment tests selected from a Quick Inventory of Depressive Symptomatology Self-Report (QIDS-SR), a Hamilton Anxiety Rating Scale (HAM-A), or a Hamilton Depression Rating Scale (HAM-D).

Another embodiment described herein is a method for treating a subject suffering from, or at risk of suffering from, depression, major depressive disorder, anxiety, or combination thereof by characterizing the gut microbiome metabolic profile of the subject, the method comprising: (a) obtaining one or more biological samples from the subject; (b) measuring a baseline concentration of one or more tryptophan gut metabolites selected from indoxyl sulfate, indole-3-propionic acid, 5-methoxytryptamine, indole-3-acetate, indole-3-lactate, or combinations thereof; (c) determining baseline ratios of indoxyl sulfate concentration to the concentrations of indole-3-propionic acid, 5-methoxytryptamine, indole-3-acetate, and indole-3-lactate, wherein increased baseline ratios of indoxyl sulfate indicate that the subject is suffering from, or at risk of suffering from, depression, major depressive disorder, anxiety or combination thereof; and (d) administering a therapy to the subject when increased ratios of indoxyl sulfate are determined. In one aspect, the subject is undergoing cognitive behavioral therapy, treatment with an anti-depressant, or a combination thereof. In another aspect, the anti-depressant comprises one or more of selective serotonin reuptake inhibitors (SSRI), tricyclic anti-depressants (TCA), selective serotonin and norepinephrine reuptake inhibitors (SNRI), monoamine oxidase inhibitors (MAOI), anxiolytics, antipsychotics, or combinations thereof. In another aspect, the anti-depressant comprises one or more of citalopram (Celexa®), escitalopram (Lexapro®), duloxetine (Cymbalta®), fluoxetine (Prozac®), paroxetine (Paxil®), sertraline (Zoloft®), trazodone (Desyrel®), lorazepam (Ativan®), oxazepam (Serax®), aripiprazole (Abilify®), clozapine (Clozaril®), haloperidol (Haldol®), olanzapine (Zyprexa®), quetiapine (Seroquel®), risperidone (Risperdal®), ziprasidone (Geodon®), amitriptyline, amoxapine, desipramine (Norpramin®), doxepin, imipramine (Tofranil®), nortriptyline (Pamelor®), protriptyline, trimipramine, or combinations thereof. In another aspect, the anti-depressant comprises escitalopram (Lexapro®), duloxetine (Cymbalta®), or a combination thereof. In another aspect, the measuring the concentration of one or more tryptophan gut metabolites is repeated at least twice, wherein a first measurement is taken as a baseline reading and a second or subsequent measurement is taken following administration of the therapy to the subject. In another aspect, the measured baseline concentrations and determined baseline ratios are compared to a healthy control. In another aspect, the therapy comprises one or more therapeutic agents to trap and sequester indoxyl sulfate. In another aspect, the therapeutic agent comprises AB-2004 or activated charcoal. In another aspect, the therapy comprises a probiotic gut bacterial strain that produces indole-3-propionic acid and does not produce indoxyl sulfate. In another aspect, the probiotic gut bacterial strain comprises Clostridium sporogenes. In another aspect, the therapy reduces tryptophanase expressing bacteria. In another aspect, the therapy comprises one or more therapeutic agents to inhibit enzymatic activity of sulfotransferase liver enzymes that produce indoxyl sulfate. In another aspect, the therapy comprises one or more therapeutic agents to inhibit aryl hydrocarbon receptor activity of intestinal immune cells. In another aspect, the method further comprises performing functional magnetic resonance imaging of the subject prior to, during, or following administration of the therapy to the subject to monitor changes in brain resting state functional connectivity. In another aspect, the method further comprises assessing disease severity of the subject prior to, during, and following administration of the therapy to the subject. In another aspect, assessing disease severity comprises the use of one or more disease assessment tests selected from a Quick Inventory of Depressive Symptomatology Self-Report (QIDS-SR), a Hamilton Anxiety Rating Scale (HAM-A), or a Hamilton Depression Rating Scale (HAM-D).

Another embodiment described herein is a method for stratifying one or more subjects suffering from a neuropsychiatric disease, disorder, or condition, including depression, major depressive disorder, anxiety, or combinations thereof into subgroups based on individual gut microbiome composition, enzymatic activity, concentrations and ratios of metabolites produced by gut bacteria, or combinations thereof, the method comprising: (a) obtaining one or more biological samples from the subjects; (b) measuring a baseline concentration of one or more tryptophan gut metabolites selected from indoxyl sulfate, indole-3-propionic acid, 5-methoxytryptamine, indole-3-acetate, indole-3-lactate, or combinations thereof; (c) determining a baseline ratio of indoxyl sulfate concentration to concentrations of indole-3-propionic acid, 5-methoxytryptamine, indole-3-acetate, indole-3-lactate, or combinations thereof; (d) stratifying the subjects into subgroups based on the determined baseline ratios of indoxyl sulfate concentration to concentrations of indole-3-propionic acid, 5-methoxytryptamine, indole-3-acetate, indole-3-lactate, or combinations thereof; and (e) assessing disease severity of subjects in each stratified subgroup. In one aspect, increased ratios of indoxyl sulfate concentration to concentrations of indole-3-propionic acid, 5-methoxytryptamine, indole-3-acetate, and indole-3-lactate indicate that the subject is suffering from, or at risk of suffering from, depression, major depressive disorder, anxiety, or combinations thereof. In another aspect, the measured baseline concentrations and determined baseline ratios are compared to a healthy control. In another aspect, assessing disease severity of subjects in each stratified subgroup comprises using one or more disease assessment tests selected from a Quick Inventory of Depressive Symptomatology Self-Report (QIDS-SR), a Hamilton Anxiety Rating Scale (HAM-A), or a Hamilton Depression Rating Scale (HAM-D). In another aspect, the method further comprises administering a therapy to subjects in each stratified subgroup based on the assessed disease severity and the determined baseline ratios of indoxyl sulfate concentration to concentrations of indole-3-propionic acid, 5-methoxytryptamine, indole-3-acetate, indole-3-lactate, or combinations thereof. In another aspect, the therapy comprises modulating the gut microbiome composition or enzymatic activity of the subject by: (a) increasing the level of indole-3-propionic acid producing bacteria; (b) decreasing the level of indoxyl sulfate producing bacteria; (c) inhibiting bacterial enzymes that produce indoxyl sulfate; (d) activating bacterial enzymes that produce indole-3-propionic acid; or a combination of any of (a)-(d).

Another embodiment described herein is the use of a therapeutic agent to trap and sequester indoxyl sulfate for the treatment of a subject suffering from, or at risk of suffering from, depression, major depressive disorder, anxiety, or combinations thereof.

Another embodiment described herein is the use of an indole-3-propionic acid producing probiotic gut bacterial strain for the treatment of a subject suffering from, or at risk of suffering from, depression, major depressive disorder, anxiety, or combinations thereof. In one aspect, the probiotic gut bacterial strain comprises Clostridium sporogenes.

Another embodiment described herein is the use of a therapeutic agent to inhibit enzymatic activity of sulfotransferase liver enzymes that produce indoxyl sulfate for the treatment of a subject suffering from, or at risk of suffering from, depression, major depressive disorder, anxiety, or combinations thereof.

Another embodiment described herein is the use of a therapeutic agent to inhibit aryl hydrocarbon receptor activity of intestinal immune cells for the treatment of a subject suffering from, or at risk of suffering from, depression, major depressive disorder, anxiety, or combinations thereof.

Another embodiment described herein is a method for treating a subject suffering from, or at risk of suffering from, immune metabolic depression, anxiety, or combinations thereof by characterizing the gut microbiome metabolic profile of the subject, the method comprising: (a) obtaining one or more biological samples from the subject; (b) measuring a concentration of one or more tryptophan gut metabolites selected from indoxyl sulfate, indole-3-propionic acid, 5-methoxytryptamine, indole-3-acetate, indole-3-lactate, or combinations thereof; (c) determining ratios of indoxyl sulfate concentration to the concentrations of indole-3-propionic acid, 5-methoxytryptamine, indole-3-acetate, indole-3-lactate, or combinations thereof, wherein increased ratios of indoxyl sulfate indicate that the subject is suffering from, or at risk of suffering from, depression, major depressive disorder, anxiety, or combinations thereof; (d) measuring aryl hydrocarbon receptor activity of intestinal immune cells; and (e) administering a therapy to the subject when increased ratios of indoxyl sulfate are determined. In one aspect, the measured concentrations and determined ratios are compared to a healthy control. In another aspect, the therapy comprises modulating the gut microbiome composition or enzymatic activity of the subject by: (a) increasing the level of indole-3-propionic acid producing bacteria; (b) decreasing the level of indoxyl sulfate producing bacteria; (c) inhibiting bacterial enzymes that produce indoxyl sulfate; (d) activating bacterial enzymes that produce indole-3-propionic acid; or a combination of any of (a)-(d). In another aspect, the therapy comprises one or more therapeutic agents to inhibit aryl hydrocarbon receptor activity of intestinal immune cells.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:

Clause 1. A method for treating a subject suffering from, or at risk of suffering from, depression, major depressive disorder, anxiety, or a combination thereof by modulating the gut microbiome composition or enzymatic activity of the subject, the method comprising:
(a) increasing the level of indole-3-propionic acid producing bacteria;
(b) decreasing the level of indoxyl sulfate producing bacteria;
(c) inhibiting bacterial enzymes that produce indoxyl sulfate;
(d) activating bacterial enzymes that produce indole-3-propionic acid; or
a combination of any of (a)-(d).

Clause 2. The method of clause 1, wherein the subject is undergoing cognitive behavioral therapy, treatment with an anti-depressant, or a combination thereof.

Clause 3. The method of clause 1 or 2, wherein the anti-depressant comprises one or more of selective serotonin reuptake inhibitors (SSRI), tricyclic anti-depressants (TCA), selective serotonin and norepinephrine reuptake inhibitors (SNRI), monoamine oxidase inhibitors (MAOI), anxiolytics, antipsychotics, or combinations thereof.

Clause 4. The method of any one of clauses 1-3, wherein the anti-depressant comprises one or more of citalopram (Celexa®), escitalopram (Lexapro®), duloxetine (Cymbalta®), fluoxetine (ProzadD), paroxetine (Paxil®), sertraline (Zoloft®), trazodone (Desyrel®)), lorazepam (Ativan®), oxazepam (Serax®), aripiprazole (Abilify®), cozapine (Clozaril®), haloperidol (Haldol®), olanzapine (Zyprexa®), quetiapine (Seroquel®), risperidone (Risperdal®), ziprasidone (Geodon®), amitriptyline, amoxapine, desipramine (Norpramin®), doxepin, imipramine (Tofranil®), nortriptyline (Pamelor®), protriptyline, trimipramine, or combinations thereof.

Clause 5. The method of any one of clauses 1-4, wherein the anti-depressant comprises escitalopram (Lexapro®), duloxetine (Cymbalta®), or a combination thereof.

Clause 6. The method of any one of clauses 1-1, wherein the indole-3-propionic acid producing bacteria comprise Clostridium sporogenes.

Clause 7. The method of any one of clauses 1-6, wherein increasing the level of indole-3-propionic acid producing bacteria comprises obtaining and administering to the subject probiotic Clostridium sporogenes.

Clause 8. The method of any one of clauses 1-1, wherein the indoxyl sulfate producing bacteria comprise tryptophanase expressing bacteria.

Clause 9. The method of any one of clauses 1-1, further comprising administering to the subject one or more therapeutic agents to trap and sequester indoxyl sulfate.

Clause 10. The method of any one of clauses 1-9, wherein the therapeutic agent comprises AB-2004 or activated charcoal.

Clause 11. The method of any one of clauses 1-10, further comprising administering to the subject one or more therapeutic agents to inhibit enzymatic activity of sulfotransferase liver enzymes that produce indoxyl sulfate.

Clause 12. The method of any one of clauses 1-11, wherein the method prevents indoxyl sulfate from crossing the blood-brain barrier.

Clause 13. The method of any one of clauses 1-12, further comprising administering to the subject one or more therapeutic agents to inhibit aryl hydrocarbon receptor activity of intestinal immune cells.

Clause 14. The method of any one of clauses 1-13, further comprising:
measuring a concentration of one or more of the subject's tryptophan gut metabolites comprising one or more of indoxyl sulfate, indole-3-propionic acid, 5-methoxytryptamine, indole-3-acetate, indole-3-lactate, or combinations thereof;
and determining ratios of indoxyl sulfate concentration to the concentrations of indole-3-propionic acid, 5-methoxytryptamine, indole-3-acetate, or indole-3-lactate.

Clause 15. The method of any one of clauses 1-14, wherein the measuring the concentration of one or more tryptophan gut metabolites is repeated at least twice; wherein a first measurement is taken as a baseline reading and a second or subsequent measurement is taken following modulation of the gut microbiome composition, enzymatic activity, or combination thereof of the subject.

Clause 16. The method of any one of clauses 1-15, wherein the measured concentrations and determined ratios are compared to a healthy control.

Clause 17. The method of any one of clauses 1-16, wherein ratios are correlated with Hamilton Anxiety scores, Hamilton Depression scores, or Quick Inventory of Depressive Symptoms.

Clause 18. The method of any one of clauses 1-17, wherein the method modulates a concentration of one or more tryptophan gut metabolites in the subject selected from indoxyl sulfate, indole-3-propionic acid, 5-methoxytryptamine, indole-3-acetate, indole-3-lactate, or combinations thereof.

Clause 19. The method of any one of clauses 1-18, wherein the method reduces the concentration of indoxyl sulfate in the subject.

Clause 20. The method of any one of clauses 1-19, wherein the method increases the concentration of indole-3-propionic acid in the subject.

Clause 21. The method of any one of clauses 1-20, further comprising assessing disease severity of the subject prior to, during, or following modulation of the gut microbiome composition, enzymatic activity, or combination thereof of the subject.

Clause 22. The method of any one of clauses 1-21, wherein assessing disease severity comprises the use of one or more disease assessment tests selected from a Quick Inventory of Depressive Symptomatology Self-Report (QIDS-SR), a Hamilton Anxiety Rating Scale (HAM-A), or a Hamilton Depression Rating Scale (HAM-D).

Clause 23. A method for treating a subject suffering from, or at risk of suffering from, depression, major depressive disorder, anxiety, or combination thereof by characterizing the gut microbiome metabolic profile of the subject, the method comprising:
(a) obtaining one or more biological samples from the subject;
(b) measuring a baseline concentration of one or more tryptophan gut metabolites selected from indoxyl sulfate, indole-3-propionic acid, 5-methoxytryptamine, indole-3-acetate, indole-3-lactate, or combinations thereof;
(c) determining baseline ratios of indoxyl sulfate concentration to the concentrations of indole-3-propionic acid, 5-methoxytryptamine, indole-3-acetate, and indole-3-lactate, wherein increased baseline ratios of indoxyl sulfate indicate that the subject is suffering from, or at risk of suffering from, depression, major depressive disorder, anxiety or combination thereof; and
(d) administering a therapy to the subject when increased ratios of indoxyl sulfate are determined.

Clause 24. The method of clause 23, wherein the subject is undergoing cognitive behavioral therapy, treatment with an anti-depressant, or a combination thereof.

Clause 25. The method of clause 23 o4 24, wherein the anti-depressant comprises one or more of selective serotonin reuptake inhibitors (SSRI), tricyclic anti-depressants (TCA), selective serotonin and norepinephrine reuptake inhibitors (SNRI), monoamine oxidase inhibitors (MAOI), anxiolytics, antipsychotics, or combinations thereof.

Clause 26. The method of any one of clauses 23-25, wherein the anti-depressant comprises one or more of citalopram (Celexa®), escitalopram (Lexapro®), duloxetine (Cymbalta®), fluoxetine (Prozac®), paroxetine (Paxil®), sertraline (Zoloft®), trazodone (Desyrel®), lorazepam (Ativan®), oxazepam (Serax®), aripiprazole (Abilify®), clozapine (Clozaril®), haloperidol (Haldol®), olanzapine (Zyprexa®), quetiapine (SeroqueW®), risperidone (Risperdal®), ziprasidone (Geodon®), amitriptyline, amoxapine, desipramine (Norpramin®), doxepin, imipramine (Tofranil®), nortriptyline (Pamelor®), protriptyline, trimipramine, or combinations thereof.

Clause 27. The method of any one of clauses 23-26, wherein the anti-depressant comprises escitalopram (Lexapro®), duloxetine (Cymbalta®), or a combination thereof.

Clause 28. The method of any one of clauses 23-27, wherein the measuring the concentration of one or more tryptophan gut metabolites is repeated at least twice, wherein a first measurement is taken as a baseline reading and a second or subsequent measurement is taken following administration of the therapy to the subject.

Clause 29. The method of any one of clauses 23-28, wherein the measured baseline concentrations and determined baseline ratios are compared to a healthy control.

Clause 30. The method of any one of clauses 23-29, wherein the therapy comprises one or more therapeutic agents to trap and sequester indoxyl sulfate.

Clause 31. The method of any one of clauses 23-30, wherein the therapeutic agent comprises AB-2004 or activated charcoal.

Clause 32. The method of any one of clauses 23-31, wherein the therapy comprises a probiotic gut bacterial strain that produces indole-3-propionic acid and does not produce indoxyl sulfate.

Clause 33. The method of any one of clauses 23-32, wherein the probiotic gut bacterial strain comprises Clostridium sporogenes.

Clause 34. The method of any one of clauses 23-33, wherein the therapy reduces tryptophanase expressing bacteria.

Clause 35. The method of any one of clauses 23-34, wherein the therapy comprises one or more therapeutic agents to inhibit enzymatic activity of sulfotransferase liver enzymes that produce indoxyl sulfate.

Clause 36. The method of any one of clauses 23-35, wherein the therapy comprises one or more therapeutic agents to inhibit aryl hydrocarbon receptor activity of intestinal immune cells.

Clause 37. The method of any one of clauses 23-36, further comprising performing functional magnetic resonance imaging of the subject prior to, during, or following administration of the therapy to the subject to monitor changes in brain resting state functional connectivity.

Clause 38. The method of any one of clauses 23-37, further comprising assessing disease severity of the subject prior to, during, and following administration of the therapy to the subject.

Clause 39. The method of any one of clauses 23-38, wherein assessing disease severity comprises the use of one or more disease assessment tests selected from a Quick Inventory of Depressive Symptomatology Self-Report (QIDS-SR), a Hamilton Anxiety Rating Scale (HAM-A), or a Hamilton Depression Rating Scale (HAM-D).

Clause 40. A method for stratifying one or more subjects suffering from a neuropsychiatric disease, disorder, or condition, including depression, major depressive disorder, anxiety, or combinations thereof into subgroups based on individual gut microbiome composition, enzymatic activity, concentrations and ratios of metabolites produced by gut bacteria, or combinations thereof, the method comprising:
(a) obtaining one or more biological samples from the subjects;
(b) measuring a baseline concentration of one or more tryptophan gut metabolites selected from indoxyl sulfate, indole-3-propionic acid, 5-methoxytryptamine, indole-3-acetate, indole-3-lactate, or combinations thereof;
(c) determining a baseline ratio of indoxyl sulfate concentration to concentrations of indole-3-propionic acid, 5-methoxytryptamine, indole-3-acetate, indole-3-lactate, or combinations thereof;
(d) stratifying the subjects into subgroups based on the determined baseline ratios of indoxyl sulfate concentration to concentrations of indole-3-propionic acid, 5-methoxytryptamine, indole-3-acetate, indole-3-lactate, or combinations thereof; and
(e) assessing disease severity of subjects in each stratified subgroup.

Clause 41. The method of clause 40, wherein increased ratios of indoxyl sulfate concentration to concentrations of indole-3-propionic acid, 5-methoxytryptamine, indole-3-acetate, and indole-3-lactate indicate that the subject is suffering from, or at risk of suffering from, depression, major depressive disorder, anxiety, or combinations thereof.

Clause 42. The method of clause 40 or 41, wherein the measured baseline concentrations and determined baseline ratios are compared to a healthy control.

Clause 43. The method of any one of clauses 40-42, wherein assessing disease severity of subjects in each stratified subgroup comprises using one or more disease assessment tests selected from a Quick Inventory of Depressive Symptomatology Self-Report (QIDS-SR), a Hamilton Anxiety Rating Scale (HAM-A), or a Hamilton Depression Rating Scale (HAM-D).

Clause 44. The method of any one of clauses 40-43, further comprising administering a therapy to subjects in each stratified subgroup based on the assessed disease severity and the determined baseline ratios of indoxyl sulfate concentration to concentrations of indole-3-propionic acid, 5-methoxytryptamine, indole-3-acetate, indole-3-lactate, or combinations thereof.

Clause 45. The method of any one of clauses 40-44, wherein the therapy comprises modulating the gut microbiome composition or enzymatic activity of the subject by:
(a) increasing the level of indole-3-propionic acid producing bacteria;
(b) decreasing the level of indoxyl sulfate producing bacteria;
(c) inhibiting bacterial enzymes that produce indoxyl sulfate;
(d) activating bacterial enzymes that produce indole-3-propionic acid; or
a combination of any of (a)-(d).

Clause 46. Use of a therapeutic agent to trap and sequester indoxyl sulfate for the treatment of a subject suffering from, or at risk of suffering from, depression, major depressive disorder, anxiety, or combinations thereof.

Clause 47. Use of an indole-3-propionic acid producing probiotic gut bacterial strain for the treatment of a subject suffering from, or at risk of suffering from, depression, major depressive disorder, anxiety, or combinations thereof.

Clause 48. The use of any one of clauses 40-47, wherein the probiotic gut bacterial strain comprises Clostridium sporogenes.

Clause 49. Use of a therapeutic agent to inhibit enzymatic activity of sulfotransferase liver enzymes that produce indoxyl sulfate for the treatment of a subject suffering from, or at risk of suffering from, depression, major depressive disorder, anxiety, or combinations thereof.

Clause 50. Use of a therapeutic agent to inhibit aryl hydrocarbon receptor activity of intestinal immune cells for the treatment of a subject suffering from, or at risk of suffering from, depression, major depressive disorder, anxiety, or combinations thereof.

Clause 51. A method for treating a subject suffering from, or at risk of suffering from, immune metabolic depression, anxiety, or combinations thereof by characterizing the gut microbiome metabolic profile of the subject, the method comprising:
(a) obtaining one or more biological samples from the subject;
(b) measuring a concentration of one or more tryptophan gut metabolites selected from indoxyl sulfate, indole-3-propionic acid, 5-methoxytryptamine, indole-3-acetate, indole-3-lactate, or combinations thereof;
(c) determining ratios of indoxyl sulfate concentration to the concentrations of indole-3-propionic acid, 5-methoxytryptamine, indole-3-acetate, indole-3-lactate, or combinations thereof, wherein increased ratios of indoxyl sulfate indicate that the subject is suffering from, or at risk of suffering from, depression, major depressive disorder, anxiety, or combinations thereof;
(d) measuring aryl hydrocarbon receptor activity of intestinal immune cells; and
(e) administering a therapy to the subject when increased ratios of indoxyl sulfate are determined.

Clause 52. The method of clause 51, wherein the measured concentrations and determined ratios are compared to a healthy control.

Clause 53. The method of clause 51 or 52, wherein the therapy comprises modulating the gut microbiome composition or enzymatic activity of the subject by:
(a) increasing the level of indole-3-propionic acid producing bacteria;
(b) decreasing the level of indoxyl sulfate producing bacteria;
(c) inhibiting bacterial enzymes that produce indoxyl sulfate;
(d) activating bacterial enzymes that produce indole-3-propionic acid; or
a combination of any of (a)-(d).

Clause 54. The method of any one of clauses 51-53, wherein the therapy comprises one or more therapeutic agents to inhibit aryl hydrocarbon receptor activity of intestinal immune cells.

### EXAMPLES

### Example 1

PReDICT Study

Baseline measurements of compounds including indole-3-acetate, indole-3-lactate, indole-3-propionic acid, indoxyl sulfate, tryptophan, and 5-methoxytryptamine were correlated with baseline Hamilton Rating Scale for Depression (HRSD) scores for male, female, and all study participants. Structures of the various indoles are shown below.

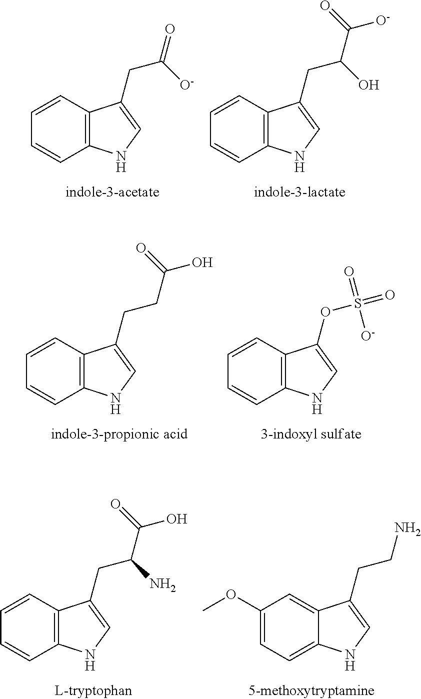

Baseline results are shown in Table 1 and FIG. 1 as a heat map of Spearman rank correlations between baseline indole intensity/ratio and baseline Hamilton Rating Scale for Depression (HRSD) score. A positive correlation (shown in red) indicates more severe depression associated with greater abundance of metabolite. Higher levels of indoxyl sulfate at baseline were found to be associated with higher levels of depression at baseline. Additionally, higher levels of indole-3-propionic acid at baseline were found to be associated with lower levels of depression at baseline.

TABLE 1
Baseline correlations with Depression (Hamilton
Rating Scale for Depression)

| | | p | r | p | r | p |
|---|---|---|---|---|---|---|
| Compound | r All | All | Female | Female | Male | Male |
| Indole-3-acetate | 0.11 | 0.13 | 0.01 | 0.89 | 0.26 | 0.03 |
| Indole-3-lactate | 0.01 | 0.93 | −0.01 | 0.87 | 0.04 | 0.72 |
| Indole-3-propionic | −0.21 | 0.00 | −0.25 | 0.01 | −0.16 | 0.17 |
| acid | | | | | | |
| Indoxyl sulfate | 0.20 | 0.00 | 0.16 | 0.09 | 0.27 | 0.02 |
| Tryptophan | −0.05 | 0.68 | −0.05 | 0.60 | −0.03 | 0.78 |
| 5-methoxytryptamine | −0.05 | 0.50 | 0.01 | 0.89 | −0.12 | 0.33 |

Bold values are signficant

Baseline measurements of compounds including indole-3-acetate, indole-3-lactate, indole-3-propionic acid, indoxyl sulfate, tryptophan, and 5-methoxytryptamine were correlated with baseline Quick Inventory of Depressive Symptomatology-Self-Rated (QIDS-SR) phenotype items for male, female, and all study participants. The 16 QIDS-SR items include:

| | |
|---|---|
| QIDS1 | Falling asleep |
| QIDS2 | Sleep during the night |
| QIDS3 | Waking up too early |
| QIDS4 | Sleeping too much |
| QIDS5 | Feeling sad |
| QIDS6 | Decreased appetite |
| QIDS7 | Increased appetite |
| QIDS8 | Decreased weight |
| QIDS9 | Increased weight |
| QIDS10 | Concentration/decision making |
| QIDS11 | View of myself |
| QIDS12 | Thoughts of death or suicide |
| QIDS13 | General interest |
| QIDS14 | Energy level |
| QIDS15 | Feeling slowed down |
| QIDS16 | Feeling restless |

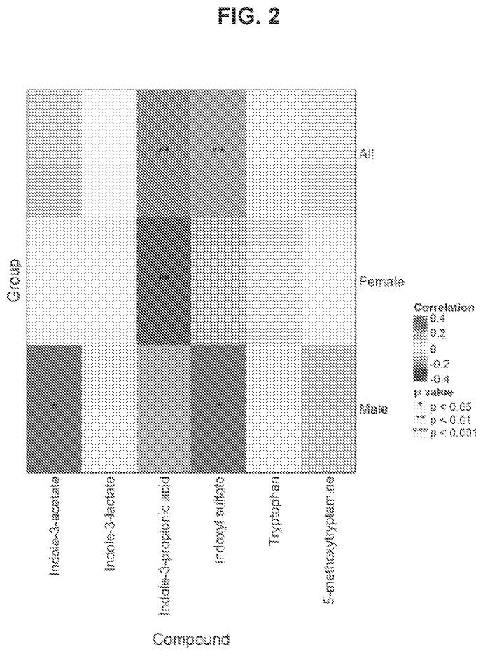

Results are shown in FIG. 2 as a heat map of Spearman rank correlations between baseline QIDS-SR items 1-16 and baseline indole intensity/ratio for female male, and all study participants. A positive correlation (shown in red) indicates more severe depression phenotype associated with greater abundance of metabolite. Higher levels of indoxyl sulfate at baseline were found to be associated with more severe symptoms of sleeping too much (QIDS 4); feeling sad (QIDS 5); decreased appetite (QIDS 6); general interest (QIDS 13); and feeling restless (QIDS 16).

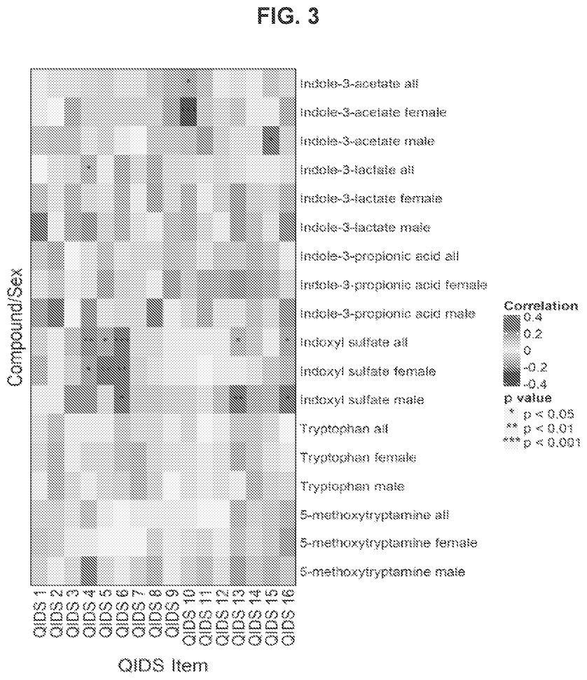

Baseline comorbidities of anxiety and depression and baseline measurements of compounds including indole-3-acetate, indole-3-lactate, indole-3-propionic acid, indoxyl sulfate, tryptophan, and 5-methoxytryptamine were compared for female, male, and all study participants. Results are shown in FIG. 3 as a heat map of Mann-Whitney U between-group comparisons between baseline comorbidities and baseline indole intensity/ratio for female, male, and all study participants. Anxious=diagnosed with anxious comorbid disorder versus not diagnosed. High/low anxiety=binary grouping based on cut-off score of 15 on Hamilton Rating Scale for Anxiety (HRSA). Depression severity=binary grouping based on cut-off score of 20 on Hamilton Rating Scale for Depression (HRSD). Fold change>1 (shown in red) indicates greater abundance of metabolite in more extreme group. Anxious versus non anxious comorbidity in females for tryptophan levels was found to have a fold change of 1.09 (p=0.04). Overall, there were not many differences observed when examining categorical groups.

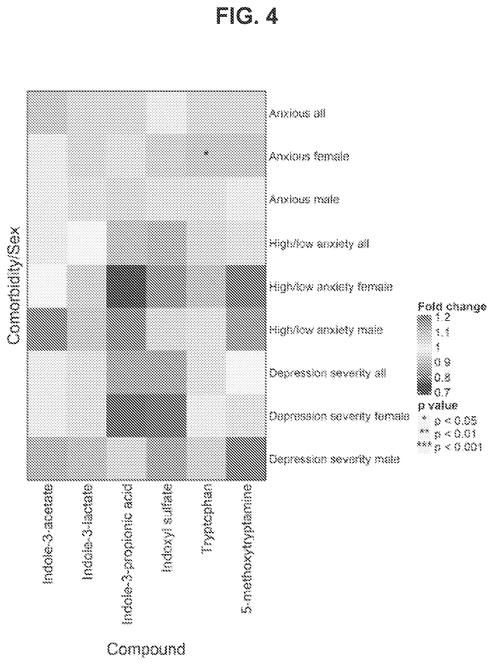

The measured levels of compounds including indole-3-acetate, indole-3-lactate, indole-3-propionic acid, indoxyl sulfate, tryptophan, and 5-methoxytryptamine at baseline and 12 weeks following different antidepressant treatments were compared for changes. Results are shown in FIG. 4 as a heat map of Spearman rank correlations between 12-week change in indole intensity/ratio and treatment groups for female, male, and all study participants. Fold change>1 (shown in red) indicates increases in metabolite from baseline to week 12. The levels of 5-methoxytryptamine were found to significantly decrease as a result of medication treatment (e.g., duloxetine or escitalopram), but not with CBT.

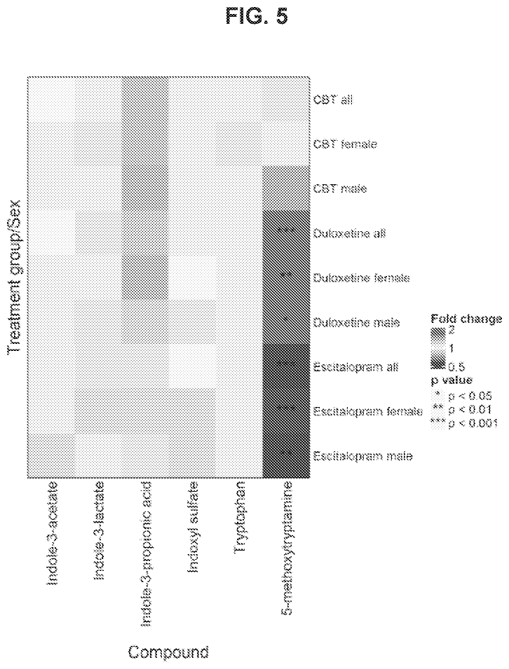

Correlations between changes in measured levels of compound metabolites and changes in depression severity from baseline to 12 weeks were assessed for female, male, and all study participants with different antidepressant treatments. Results are shown in FIG. 5 as a heat map of Spearman rank correlations between 12-week change in indole intensity/ratio and 12-week change in depression for female, male, and all study participants with different treatments. Positive correlation (shown in red) indicates improvement of depression associated with decrease of metabolite. A decrease in 5-methoxytryptamine from baseline to week 12 was found to be significantly associated with improved depression in men undergoing CBT, while an increase in 5-methoxytryptamine from baseline to week 12 is associated with improved depression in women undergoing CBT. Given the baseline associations between indoxyl sulfate, indole-3-propionic acid, and depression, it was slightly unexpected to observe no correlations of those compounds. These results suggest that the anti-depressive drugs of escitalopram or duloxetine are not effective in changing levels of indoles, and that other types of drugs which target the gut microbiome may be needed to regulate indoxyl sulfate levels.

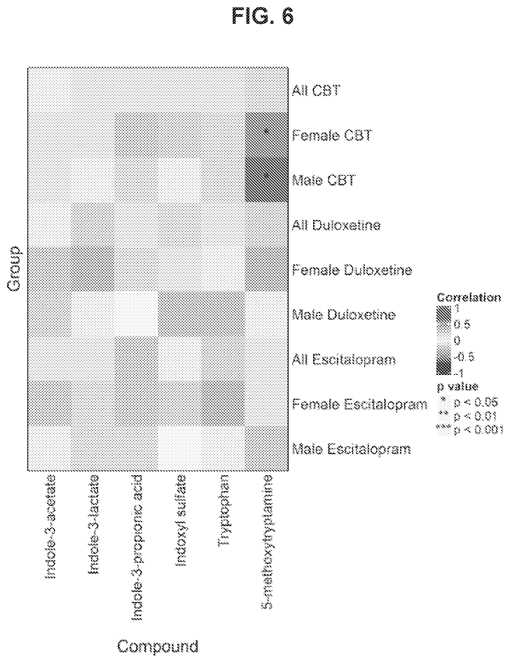

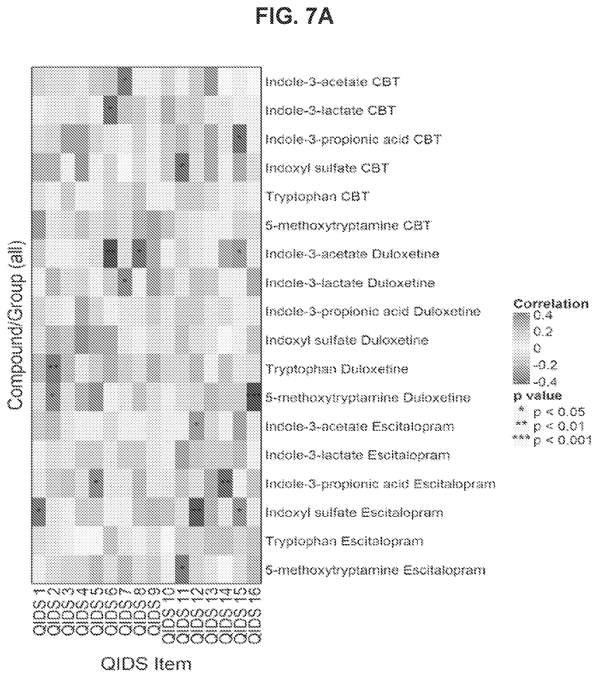

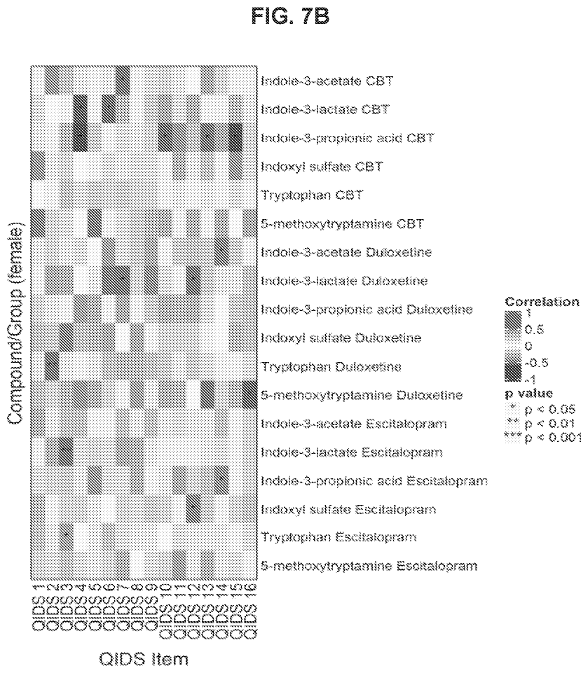

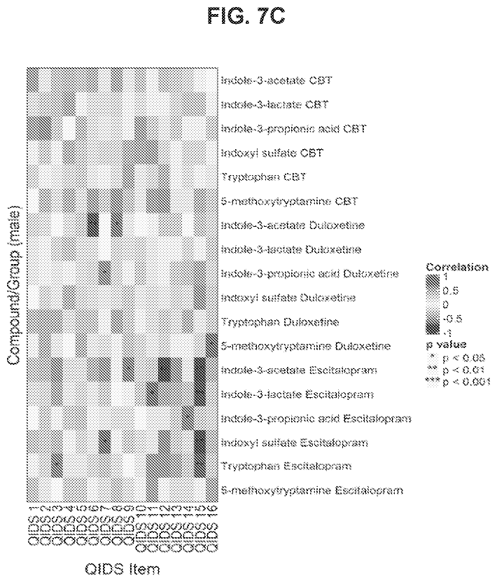

Correlations between changes in measured levels of compound metabolites and changes in specific depression phenotypes from baseline to 12 weeks were assessed for female, male, and all study participants with different antidepressant treatments. Results are shown in FIG. 6A as a heat map of Spearman rank correlations between 12-week change in indole intensity/ratio and 12-week change in depression phenotype (QIDS-SR items 1-16) for all study participants with different treatments. Results are shown in FIG. 6B as a heat map of Spearman rank correlations between 12-week change in indole intensity/ratio and 12-week change in depression phenotype (QIDS-SR items 1-16) for female study participants with different treatments. Results are shown in FIG. 6C as a heat map of Spearman rank correlations between 12-week change in indole intensity/ratio and 12-week change in depression phenotype (QIDS-SR items 1-16) for male study participants with different treatments. Positive correlation (shown in red) indicates improvement of depression phenotype associated with increase of metabolite.

For all study participants (FIG. 6A), a decrease in indoxyl sulfate from baseline to week 12 was found to be associated with alleviated thoughts of death or suicide (QIDS 12) in the group treated with escitalopram. Decreases from baseline to week 12 of indole-3-propionic acid levels were associated with alleviated feeling sad and energy level (QIDS 5 and 14) in the group treated with escitalopram, which was slightly unexpected. Additionally, decreases from baseline to week 12 of indoxyl sulfate were associated with alleviated falling asleep, thoughts of death or suicide, and feeling slowed down (QIDS 1, 12, and 15) in the group treated with escitalopram. Given baseline correlations, these findings were consistent with expected results, but the antidepressant drugs of escitalopram or duloxetine were not found to be very effective in changing the levels of indoxyl sulfate.

For female study participants (FIG. 6B), a decrease in indoxyl sulfate from baseline to week 12 was found to be associated with alleviated thoughts of death or suicide (QIDS 12) in the group treated with escitalopram. Given baseline correlations, these findings were consistent with expected results, but the antidepressant drugs of escitalopram or duloxetine were not found to be very effective in changing the levels of indoxyl sulfate. Furthermore, decreases from baseline to week 12 of indole-3-propionic acid was associated with alleviated energy level (QIDS 14) in the escitalopram group, which was slightly unexpected. Additionally, increased levels of tryptophan from baseline to week 12 were associated with alleviated sleep during the night (QIDS 2) in the group treated with duloxetine.

For male study participants (FIG. 6C), a decrease in indoxyl sulfate from baseline to week 12 was found to be associated with alleviated feeling slowed down (QIDS 15) in the group treated with escitalopram. Additionally, decreases in indole-3-acetate, indole-3-lactate, indoxyl sulfate, and tryptophan were all associated with improved energy level (QIDS 14) in the group treated with escitalopram. Overall, the escitalopram treatment group rows of the heat map appear “bluer” than the CBT or duloxetine treatment group rows, indicating that decreases in indoles are associated with improved depression phenotypes in males treated with escitalopram.

### Example 2

### Study Design

The PReDICT study protocol, clinical results and initial neuroimaging analyses have been published previously. The study was conducted through the Mood and Anxiety Disorders Program of Emory University from 2007-2013. The study was approved by Emory's Institutional Review Board. All patients provided written informed consent to participate.

PReDICT was designed to identify predictors and moderators of outcomes to three randomly assigned first-line treatments for MDD: duloxetine, escitalopram, or CBT. The study enrolled treatment-naïve adult outpatients aged 18-65 years, who had current MDD without psychotic symptoms. To be eligible for randomization, participants had to score≥18 at screening and ≥15 at baseline on the HAM-D. Key exclusion criteria included the presence of any medically significant or unstable medication condition that could impact study participation, safety, or data interpretation; any current eating disorder, obsessive-compulsive disorder, or any current substance abuse or dependence. Treatment was provided for 12 weeks with duloxetine (30-60 mg/day), escitalopram (10-20 mg/day), or CBT (16 individual one-hour sessions).

### Symptom Assessments

At the baseline visit, participants were assessed by trained interviewers using the HAM-D and the HAM-A. The HAM-A is a 14-item measure that consists of two subscales, “psychic anxiety” (items 1-6 and 14), and “somatic anxiety” (items 7-13). Psychic anxiety consists of the symptoms of anxious mood, tension, fears, depressed mood, insomnia, impaired concentration, and restlessness. Somatic anxiety consists of physical symptoms associated with the muscular, sensory, cardiovascular, respiratory, gastrointestinal, genitourinary, and autonomic systems. Participants also completed the QIDS-SR, which assesses the nine diagnostic symptom criteria for MDD. The HAM-D, HAM-A, and QIDS-SR were repeated at the Week 12 visit.

### Blood Sampling

Participants who met all eligibility criteria at the baseline visit underwent an antecubital phlebotomy, without regard for time of day or fasting/fed status. Sampling was repeated at the week 12 visit. Collected samples were allowed to clot for 20 minutes and then centrifuged at 4° C. to separate the serum, which was frozen at −80° C. until being thawed for the current analyses.

### Neuroimaging

To explore associations between indole metabolites and brain function, the resting state fMRI scans collected during the week prior to baseline were used, the details of which have previously been published. Briefly, eyes-open scanning was performed for 7.4 minutes in a 3-T Siemens TIM Trio (Siemens Medical Systems, Erlangen, Germany). Echo planar images were corrected for motion and slice-time acquisition. Scans with head motion>2 mm in any direction were removed from the analysis. The nuisance regressors, including head motion parameters, signal from the ventricle mask, and signal from a region of local white matter, were cleaned. Subsequently, data were applied a band-pass filter and smoothed using an isotropic Gaussian kernel of 8 mm full width at half maximum. The imaging anatomical and functional data sets were co-registered and normalized to standard Montreal Neurological Institute (MNI) 1-mm voxel space. Image analysis was conducted using Analysis of Functional Neurolmages (AFNI) 3dvolreg. Consistent with prior analyses, a region-of-interest seed-based approach was used to assess the resting state functional connectivity (RSFC) of the SCC. The SCC volume was defined using the Harvard-Oxford Atlas, and the SCC was thresholded at 50% probability centered on MNI coordinates 66, 24, -11. The seeds comprised two 5-mm radius spheres, with a final volume of 485 mL each. Utilizing 3dNetCorr, the mean time course of the bilateral seed was correlated voxel-wise with the rest of the brain. The voxelwise correlation coefficients were then z-scored by calculating the inverse hyperbolic tangent, yielding the seed-based RSFC maps for analysis.

### Metabolomics Data Acquisition

Metabolomics data focused on primary and polar metabolites using gas chromatography—time of flight mass spectrometry. Briefly, 30 μL of plasma was extracted at −20° C. with 1 mL degassed isopropanol/acetonitrile/water (3/3/2). Extracts were dried down, cleaned from triacylglycerides using acetonitrile/water (1/1), and derivatized with methoxyamine and trimethylsilylation. Samples (0.5 μL) were injected at 250° C. to a 30 m rtx5-SilMS column, ramped from 50-300° C. at 15° C./min, and analyzed by −70 eV electron ionization at 17 spectra/sec. Raw data were deconvoluted and processed using ChromaTOF vs. 4.1 and uploaded to the University of California, Davis BinBase database for data curation and compound identification. Result data were normalized by SERRF software to correct for drift or batch effects.

### Statistical Analyses

Indole abundance and ratios of each indole pair were included in all analyses. In order to investigate the role of indoles in depression and anxiety symptomology at baseline, partial Spearman rank correlations were conducted between the baseline abundance/ratio of each indole and HAM-D 17-item total score, HAM-A total score, HAM-A Psychic and Somatic subscores, QIDS-SR 16-item total score, and each individual QIDS-SR item after accounting for age, sex, and body mass index (BMI). Spearman correlations were also conducted between baseline indole abundance/ratio and participant demographic factors (age, BMI, height, and weight). Additionally, sex differences in baseline indole abundance/ratio were tested using Mann-Whitney U tests and fold changes in median abundance/ratio between groups.

To investigate the potential effects of treatment on indoles, changes in indole abundance from pre- to post-treatment were tested using Wilcoxon signed-rank tests and fold changes. Partial Spearman rank correlations were conducted between post-treatment indole abundance/ratio and post-treatment HAM-D 17-item total score, HAM-A total score, and HAM-A Psychic and Somatic subscores, QIDS-SR 16-item total score, and each individual QIDS-SR item, after accounting for age, sex, and baseline BMI. The same analyses were also conducted with change from pre- to post-treatment scores of all psychiatric variables, and also with fold changes from pre- to post-treatment for each indole. Additionally, differences in indole post-treatment abundance/ratio and fold change were investigated between each pair of treatment response outcome groups (treatment failure; partial response; response; remission) by conducting Mann-Whitney U tests. This analysis was also repeated with baseline indole abundances/ratios to investigate whether baseline levels of indoles may be associated with treatment outcome. All reported p-values were adjusted for multiple comparisons using the Holm method.

Neuroimaging analyses were conducted using AFNI and jamovi (www.jamovi.org). Of 122 participants who had an adequate quality of resting-state fMRI data, 80 had metabolomic measurements and clinical scores. Voxel-wise linear regression analyses were performed to examine the relationship between SCC-FC and IS or psychic anxiety scores (uncorrected p<0.005 and >250 voxels cluster size). A conjunction analysis identified overlapping areas between the SCC-FC of IS and the SCC-FC of psychic anxiety scores. Subsequently, mediation analyses were performed using the Medmod module in jamovi. Three regions identified by the whole brain linear regression analysis between SCC-FC and IS were used for the mediation analysis. Each region was explored, and combinations of the three regions, in the mediation models. For each model, the direct and indirect effects were estimated using bootstrapping with 5000 samples.

### Example 3

### Baseline Associations

Of the 344 patients randomized in PReDICT, 196 had metabolomic measures available for analysis at baseline and 127 were available at week 12. The demographic and clinical characteristics of the 196 participants are presented in Table 2.

TABLE 2
Subject Demographic and Clinical Characteristics

| Variable | N (%) (N = 196) |
|---|---|
| Sex (Female) | 122 (62.2%) |
| Race | N (%) |
| White | 73 (37.2%) |
| Native American | 58 (29.6%) |
| Black | 38 (19.4%) |
| Asian | 2 (1.0%) |
| Multiracial | 14 (7.1%) |
| Unknown/Not Reported | 11 (5.6%) |
| Hispanic ethnicity | 77 (39.3%) |
| | Mean (SD) |
| Age (years) | 39.11 (11.77) |
| Body mass index | 28.73 (6.27) |
| HAM-D 17-item total score (baseline) | 19.76 (3.77) |
| QIDS-SR total score (baseline) | 14.27 (3.83) |
| HAM-A total score (baseline) | 16.28 (5.37) |
| HAM-A psychic anxiety subscale score (baseline) | 10.79 (2.69) |
| HAM-A somatic anxiety subscale score (baseline) | 5.48 (3.73) |

Abbreviations: HAM-A: Hamilton Anxiety Rating Scale; HAM-D: Hamilton Depression Rating Scale; QIDS-SR: Quick Inventory of Depressive Symptomatology, Self-report.

Associations of Indole Metabolites with Demographic Variables

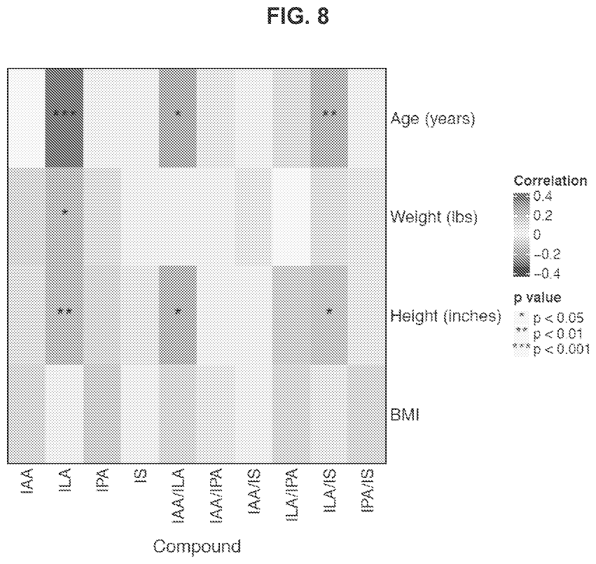

FIG. 8 shows a heat map of correlations between baseline indole abundance/ratio and participant demographic variables. Abundance of ILA was positively associated with age, height, and weight (all r-values>0.18, all p-values<0.040). The ratios of IAA/ILA (negative associations) and IS/ILA (positive associations) were also significantly associated with height and weight (all p-values<0.034). For sex differences, abundance of IAA (Fold Changes (FC)=1.19, p=0.039) and ILA (FC=1.37, p<10⁻⁹), and ratios of ILA/IPA (FC=1.40, p=0.004) and ILA/IS (FC=1.16, p=0.013) were all found to be significantly higher in men than in women.

Associations of Indole Metabolites with Depression and Anxiety

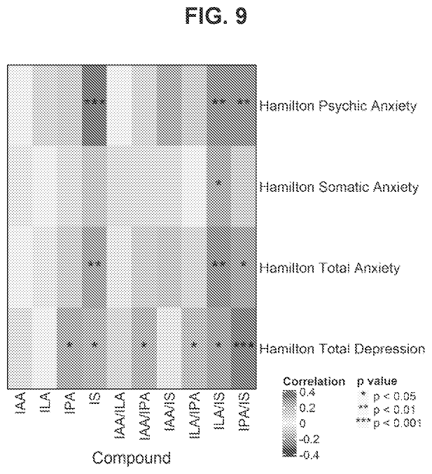

FIG. 9 shows a heat map of correlations between baseline indole abundance/ratio and baseline levels of the 17-item Hamilton Depression Rating Scale (HAM-D) total score, Hamilton Anxiety Rating Scale (HAM-A) total score, and HAM-A psychic and somatic subscores. Greater abundance of IS was associated with higher scores on the HAM-D 17-item total score (r=0.21, p=0.018), HAM-A total score (r=0.26, p=0.002), and HAM-A psychic subscore (r=0.31, p=0.0001), but not on the HAM-A somatic subscore. Additionally, the ratios of ILA/IS and IPA/IS were negatively correlated with HAM-A total and psychic scores (all rs>−0.20, all ps<0.033), for which a negative correlation indicates that increasingly severe symptoms are associated with a relative increase in IS and/or a relative decrease in ILA or IPA. Additionally, IPA/IS was negatively correlated with HAM-D total score (r=−0.24, p=0.001).

Associations of Indole Metabolites with Individual Symptoms of Depression

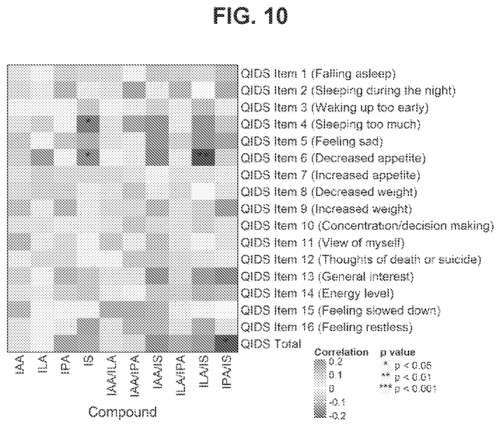

Correlations between Quick Inventory of Depressive Symptoms-Self-Report (QIDS-SR) items, and total scores and indole abundances/ratios are presented in FIG. 10. Of note, IS positively correlated with items 4 (hypersomnia; r=0.22, p=0.016) and 6 (decreased appetite;r=0.20, p=0.034), and the IPA/IS ratio negatively correlated with QIDS-SR total score (r=−0.21, p=0.027).

### Example 4

### Treatment Effects

Compound abundance significantly increased from pre- to post-treatment for ILA (FC=1.05, p=0.006), but not for any other compound/ratio (all p-values>0.12). This indicates that the treatments had limited overall effect on indole composition and levels.

For post-treatment indole abundances and ratios, there were significant correlations between IAA/IS and QIDS-SR item 15 (feeling slowed down; r=0.30, p=0.007). Additionally, post-treatment ILA abundance was significantly higher in men than in women (FC=1.24, p=0.00001), as was the ILA/IPA ratio (FC=1.54, p=0.002). Conversely, the IPA/IS ratio was lower in men than in women (FC=0.80, p=0.048). No other significant post-treatment effects were observed.

For fold changes, change in IAA/IS ratio correlated with post-treatment scores of QIDS-SR item 5 (feeling sad; r=0.27, p=0.032). No significant associations were observed when correlating indole fold changes with any other post-treatment scores, or with change in depression/anxiety scores (all p-values>0.10). Additionally, no sex differences were observed for fold changes (all p-values>0.07), and no differences in fold changes were observed between response outcome groups (all p-values>0.12).

Baseline levels of indoles and their ratios did not significantly correlate with changes in symptoms for any measure or item (all r-values<0.15, all p-values>0.59). Comparison of categorical response outcomes also showed no meaningful differences in baseline indole abundances or ratios. These analyses indicate that pre-treatment indole compound abundances are not predictive of eventual treatment outcomes.

### Example 5

Associations of Indole Metabolites with Brain Resting State Functional Connectivity

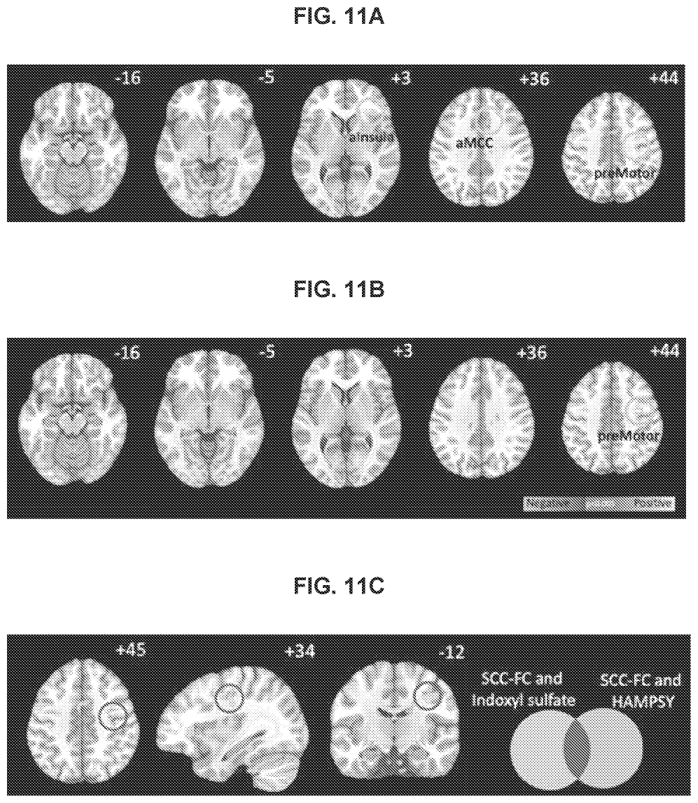

Relationships of Subcallosal Cingulate Cortex-Functional Connectivity (SCC-FC) with IS and with psychic anxiety scores are shown in FIG. 11A-C. IS abundance was positively correlated with SCC-FC with the bilateral anterior insula, anterior midcingulate cortex (aMCC), supplementary motor area (SMA), and right premotor area (FIG. 11A). Psychic anxiety scores showed a significant positive correlation with SCC-FC with the left aMCC, right precuneus, and right premotor area; there was a negative correlation with SCC-FC with the ventromedial prefrontal cortex, right orbitofrontal, and left Brodmann Area 47 (FIG. 11B). The conjunction analysis identified one overlapping area: the right premotor region (FIG. 11C).

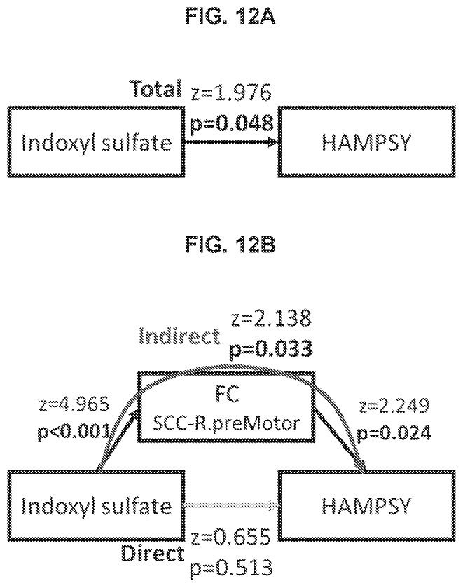

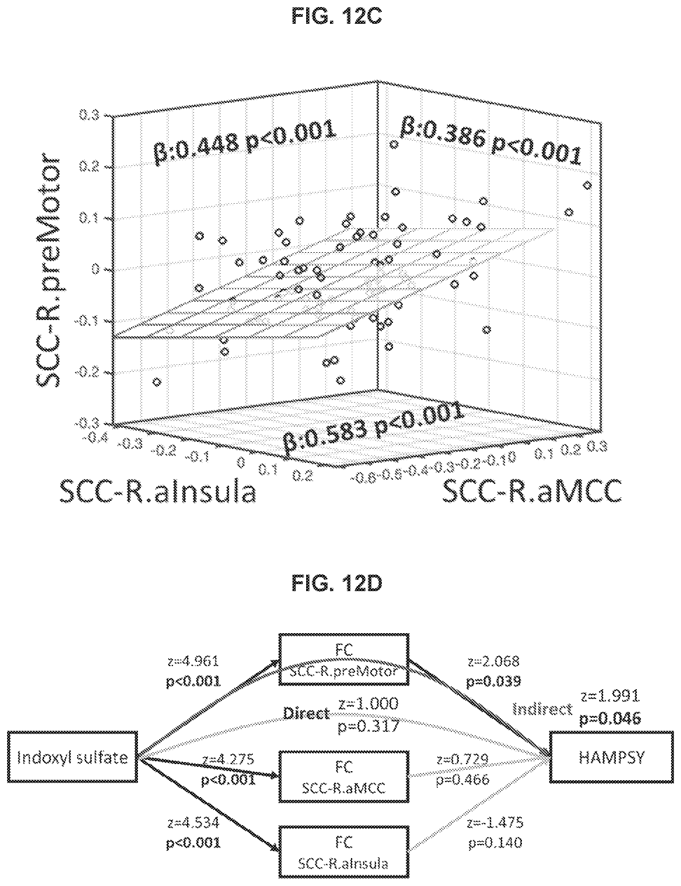

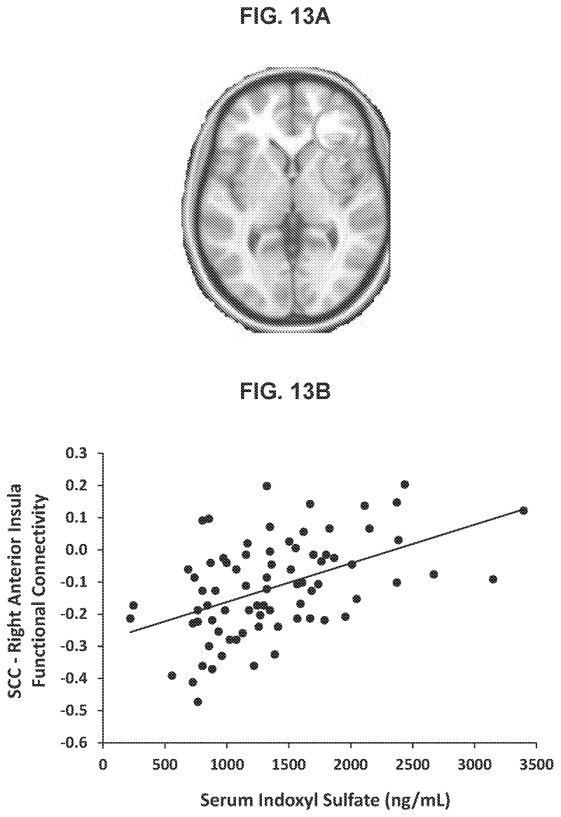

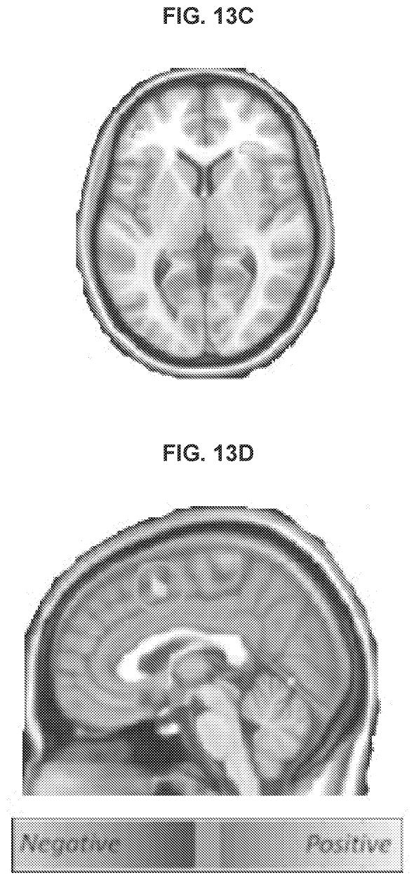

FIG. 12A-D show the mediation analyses explored whether the association of IS with psychic anxiety was mediated through its effects on SCC-FC. FIG. 12A shows the overall association between IS and psychic anxiety (z=1.976, p=0.048). FIG. 12B shows that the identified overlapping area in the SCC-FC analyses—the right premotor region—mediated the association between IS and psychic anxiety (indirect pathway: z=2.138, p=0.033). Because whole brain SCC-FC analyses also found IS concentrations to be significantly associated with two other regions previously identified in neuroimaging studies of anxiety (the right anterior insula and the aMCC, FIG. 11A), further mediation analyses were conducted incorporating these two regions along with the right premotor region. Even though the three regions were highly correlated with each other in their functional connectivity to SCC (FIG. 12C), only the right premotor region mediated the relationship between IS and psychic anxiety scores when all three regions were included in the model (FIG. 12D, indirect pathway: z=1.991, p=0.046).

Increasing evidence suggests that gut bacteria can complement human metabolism, and that together they define the metabolome comprised of the collection of small molecules in blood and in different organs. Bacteria can further metabolize compounds available through human metabolism, food-intake, and/or human ingestion of chemicals. Also, humans can further metabolize compounds produced by bacteria, which results in human-bacteria co-metabolism and the production of a large number of chemicals that can impact human health, including brain function. Examples include the metabolism of cholesterol and its clearance mediated by bacteria, which can produce secondary bile acids that were recently implicated in the pathogenesis of Alzheimer's disease. Several compounds produced from the metabolism of phospholipids and choline by gut bacteria lead to compounds like trimethylamine-N-oxide, which have been implicated in cardiovascular diabetes and CNS disease.

Indoles represent a class of gut bacterially derived compounds that are produced from tryptophan, an essential amino acid that can also be converted (through separate pathways) into tryptamine, serotonin, skatol, and melatonin, among other metabolites involved in CNS functioning and diseases. Mounting evidence suggests that indoles derived from gut bacterial metabolism exert significant biological effects and may contribute to the etiology of cardiovascular, metabolic, and psychiatric diseases. To date, research in this area has been mainly limited to experimental studies in model systems.

In this investigation, the levels of four indoles produced by gut bacteria were interrogated for their relationship to anxiety and depression severity and response to treatment. At baseline, IS abundance was found to positively correlate with severity of Psychic Anxiety and total anxiety. IPA seems protective, as noted earlier, indicating that indoles, as a class, can have mixed effects on neuropsychiatric health. Different strains of bacteria can lead to the production of different indoles; for example, tryptophanase-producing bacteria produce toxic IS while Clostridium sporogenes and other bacterial strains produce protective IPA. Notably, IS levels did not meaningfully change with treatment, and changes in IS were not correlated with improvement in depression or anxiety measures. This suggests that gut microbiome composition and activity might be modulated as an approach for developing additional classes of therapies effective in the treatment of anxiety and depression.

This association between IS and anxiety seems to be mediated by the impact of IS on the functional connectivity between the SCC and the right premotor region. IS abundances were also associated with activation of a well-established network for the processing and control of emotionally salient, particularly aversive, stimuli, comprising the anterior insula and aMCC. Taken together, these results suggest that the co-metabolism of tryptophan by certain gut microbiota that result in the production of IS, which can induce anxiety through the activation of established brain networks, and that existing treatments do not specifically resolve this pathogenetic process when they lead to clinical improvement.

The neuroimaging analyses indicate that the effect of IS on psychic anxiety symptoms is mediated through the functional connectivity of the SCC with the premotor cortex, as part of a network involved in processing emotionally salient stimuli. The anterior insula, aMCC, and supplementary motor area form a network that is involved in the attention to, interpretation of, and control of emotional responses. The premotor cortex is functionally and structurally connected to the SMA and the aMCC, which act together in the preparation and readiness for voluntary movement in response to internal and external stimuli, and the aMCC is a site of integration for the processing of pain and motor control. Outputs from this network include projections to the spinal cord and adrenal medulla, which may contribute to the sympathetic arousal and heightened cortisol release under situations of psychic stress.

Although the activity of the premotor cortex has not been a major focus in studies of anxiety and depression, using the electroencephalography measure of contingent negative variation (which localizes to the premotor cortex), it has previously been demonstrated that there is abnormal activation of this region in anxious MDD patients compared to MDD patients with psychomotor retardation. It has also been found that patients with generalized anxiety disorder have increased resting state functional connectivity between the habenula and right premotor cortex. Others have found abnormal premotor function in social anxiety disorder.

The finding of an association between IS and activation of the insula bilaterally is consistent with the insula's known involvement in processes relevant to anxiety, including emotional salience, empathy for others' pain, and processing of uncertainty. In contrast, an association between IS abundances and somatic anxiety scores were not found, nor was there an association of IS abundances with functional connectivity of the SCC-posterior insula, the insular region involved in sensorimotor integration. This reveals the specificity of the IS-anterior insula association for psychic anxiety.

Conceptualizing psychic anxiety as a chronic aversive stimulus akin to long-term pain may explain the positive correlation between higher IS levels and the QIDS-SR loss of appetite item. In mice, inflammatory pain is inhibited in the presence of hunger, mediated by neuropeptide Y signaling in the parabrachial nucleus. The association of higher IS concentrations with both reduction in appetite and increased connectivity between brain regions involved in pain processing (anterior insula and aMCC) may indicate that the symptom of low appetite reflects a compensatory response to this chronic anxiety-type pain.

Limitations of this study include the absence of a healthy control comparison group. Additionally, fecal samples were lacking, which could have been analyzed to allow a more direct correlation between specific gut microbiome species and the IS measures. It could not be determined whether IS is the etiological agent of the anxiety because IS also acts to reduce the integrity of the blood brain barrier, thereby creating the possibility that CNS penetration by an alternative molecule in the periphery is responsible for the observed association between anxiety and IS.

Taken together, the results indicate that increases in IS lead to the activation of an established network that is involved in the processing and control of aversive stimuli, but that the conscious experience of anxiety depends upon the degree of IS-related activation of SCC-right premotor cortex functional connectivity. The absence of an association between psychic anxiety scores and anterior insula/aMCC SCC-FC (FIG. 11B) may indicate that although IS activates this control network in all patients, it is only when network function is inadequate that psychic anxiety ensues in conjunction with premotor activation in preparation for action. These analyses reveal the potential of integrated peripheral metabolomic-neuroimaging analyses to reveal mechanistic pathways that are associated with neuropsychiatric symptoms, especially for characterizing the pathological impact of specific gut microbiome-derived metabolites.

### Example 6

### Patient Stratification Analysis Based on Indole Levels

Patients enrolled in the PReDICT Study were stratified based on levels of IS at baseline and ratios of IS to other indoles or based on levels of other indoles. The level of disease severity was checked in each quartile. Stratified results are as follows, comparing quartile 4 (Q4; highest level of compound) to quartile 1 (Q1; lowest level of compound):

| | |
|---|---|
| Indole-3-acetate | None |
| Indole-3-lactate | None |
| Indole-3- | QIDS was lower in Q4 than Q1 (p = 0.012) |
| propionic acid | |
| Indoxyl sulfate | HAM-A total is higher in Q4 than Q1 (p = 0.00085); |
| | Psychic anxiety is higher in Q4 than Q1 (p = 0.00024) |
| Acetate/Lactate | None |
| Acetate/ | HAM-D total is higher in Q4 than Q1 (p = 0.0123) |
| propionic acid | |
| Acetate/sulfate | Psychic anxiety is lower in Q4 than Q1 (p = 0.023); |
| | QIDS is lower in Q4 than in Q1 (p = 0.021) |
| Lactate/ | QIDS is higher in Q4 than in Q1 (p = 0.0069); Non- |
| propionic acid | anxious depression is higher in Q4 than in Q1 (p = |
| | 0.024) |
| Lactate/indoxyl | HAM-A total is lower in Q4 than Q1 (p = 0.0012); |
| sulfate | Somatic anxiety is lower in Q4 than Q1 (p = 0.011); |
| | Psychic anxiety is lower in Q4 than in Q1 (p = |
| | 0.0036); IMD is lower in Q4 than in Q1 (p = 0.01480) |
| Propionic acid/ | HAM-D total is lower in Q4 than in Q1 (p = 0.0021); |
| Indoxyl sulfate | Psychic anxiety is lower in Q4 than in Q1 (p = |
| | 0.013); QIDS is lower in Q4 than in Q1 (p = 0.0022); |
| | IMD is lower in Q4 than in Q1 (p = 0.029) |

In summary, people with the highest levels of IS do poorly and have more severe disease. Other indoles seem to be not as important, although the propionic acid/sulfate ratio does show several important differences. Such ratios among others can enable stratification of patients with anxiety disorders to enable treatment approaches based on gut microbiome modulation.

### Example 7

Patients with Immune Metabolic Disease have Higher Levels of IS

In the Netherlands Study of Depression and Anxiety (NESDA), an immune metabolic depression type has been identified. The atypical energy-related symptom dimension was linked to poorer immune-inflammatory and metabolic health, while the melancholic symptom dimension was linked to relatively better metabolic health. Persons with high atypical energy-related symptom burden, representing an immuno-metabolic depression, may be the most important group to target in prevention programs for cardiometabolic disease, and may benefit most from treatments targeting immuno-metabolic pathways. A positive association between levels of IS and symptom severity of this immune metabolic depression type in PReDICT patients was discovered, after accounting for age, sex, and BMI (r=0.22, p=0.004). No other indole compound or ratio was associated with immuno-metabolic depression. This suggests that treatments based on IS modulation might be beneficial for this type of depression. Part of the characterization of this group involves measures of IS and cytokines and testing for activation of aryl hydrocarbon receptor that connects indole and immune functions.

What is claimed:

1. A method for treating a subject suffering from, or at risk of suffering from, depression, major depressive disorder, anxiety, or a combination thereof by modulating the gut microbiome composition or enzymatic activity of the subject, the method comprising:
(a) increasing the level of indole-3-propionic acid producing bacteria;
(b) decreasing the level of indoxyl sulfate producing bacteria;
(c) inhibiting bacterial enzymes that produce indoxyl sulfate;
(d) activating bacterial enzymes that produce indole-3-propionic acid; or
a combination of any of (a)-(d).

2. The method of claim 1, wherein the subject is undergoing cognitive behavioral therapy, treatment with an anti-depressant, or a combination thereof.

3. The method of claim 2, wherein the anti-depressant comprises one or more of selective serotonin reuptake inhibitors (SSRI), tricyclic anti-depressants (TCA), selective serotonin and norepinephrine reuptake inhibitors (SNRI), monoamine oxidase inhibitors (MAOI), anxiolytics, antipsychotics, or combinations thereof.

4. The method of claim 3, wherein the anti-depressant comprises one or more of citalopram (Celexa®), escitalopram (Lexapro®), duloxetine (Cymbalta®), fluoxetine (Prozac®), paroxetine (Paxil®), sertraline (Zoloft®), trazodone (Desyrel®), lorazepam (Ativan®), oxazepam (Serax®), aripiprazole (Abilify®), clozapine (Clozaril®), haloperidol (Haldow®), olanzapine (Zyprexa®), quetiapine (Seroquel®), risperidone (Risperdal®), ziprasidone (Geodon®), amitriptyline, amoxapine, desipramine (Norpramin®), doxepin, imipramine (Tofranil®), nortriptyline (Pamelor®), protriptyline, trimipramine, or combinations thereof.

5. The method of claim 4, wherein the anti-depressant comprises escitalopram (Lexapro®), duloxetine (Cymbalta®), or a combination thereof.

6. The method of claim 1, wherein the indole-3-propionic acid producing bacteria comprise Clostridium sporogenes.

7. The method of claim 6, wherein increasing the level of indole-3-propionic acid producing bacteria comprises obtaining and administering to the subject probiotic Clostridium sporogenes.

8. The method of claim 1, wherein the indoxyl sulfate producing bacteria comprise tryptophanase expressing bacteria.

9. The method of claim 1, further comprising administering to the subject one or more therapeutic agents to trap and sequester indoxyl sulfate.

10. The method of claim 9, wherein the therapeutic agent comprises AB-2004 or activated charcoal.

11. The method of claim 1, further comprising administering to the subject one or more therapeutic agents to inhibit enzymatic activity of sulfotransferase liver enzymes that produce indoxyl sulfate.

12. The method of claim 11, wherein the method prevents indoxyl sulfate from crossing the blood-brain barrier.

13. The method of claim 1, further comprising administering to the subject one or more therapeutic agents to inhibit aryl hydrocarbon receptor activity of intestinal immune cells.

14. The method of claim 1, further comprising:
measuring a concentration of one or more of the subject's tryptophan gut metabolites comprising one or more of indoxyl sulfate, indole-3-propionic acid, 5-methoxytryptamine, indole-3-acetate, indole-3-lactate, or combinations thereof; and
determining ratios of indoxyl sulfate concentration to the concentrations of indole-3-propionic acid, 5-methoxytryptamine, indole-3-acetate, or indole-3-lactate.

15. The method of claim 14, wherein the measuring the concentration of one or more tryptophan gut metabolites is repeated at least twice; wherein a first measurement is taken as a baseline reading and a second or subsequent measurement is taken following modulation of the gut microbiome composition, enzymatic activity, or combination thereof of the subject.

16. The method of claim 14, wherein the measured concentrations and determined ratios are compared to a healthy control.

17. The method of claim 14, wherein ratios are correlated with Hamilton Anxiety scores, Hamilton Depression scores, or Quick Inventory of Depressive Symptoms.

18. The method of claim 1, wherein the method modulates a concentration of one or more tryptophan gut metabolites in the subject selected from indoxyl sulfate, indole-3-propionic acid, 5-methoxytryptamine, indole-3-acetate, indole-3-lactate, or combinations thereof.

19. The method of claim 18, wherein the method reduces the concentration of indoxyl sulfate in the subject.

20. The method of claim 18, wherein the method increases the concentration of indole-3-propionic acid in the subject.

21. The method of claim 1, further comprising assessing disease severity of the subject prior to, during, or following modulation of the gut microbiome composition, enzymatic activity, or combination thereof of the subject.

22. The method of claim 21, wherein assessing disease severity comprises the use of one or more disease assessment tests selected from a Quick Inventory of Depressive Symptomatology Self-Report (QIDS-SR), a Hamilton Anxiety Rating Scale (HAM-A), or a Hamilton Depression Rating Scale (HAM-D).

23. A method for treating a subject suffering from, or at risk of suffering from, depression, major depressive disorder, anxiety, or combination thereof by characterizing the gut microbiome metabolic profile of the subject, the method comprising:
(a) obtaining one or more biological samples from the subject;
(b) measuring a baseline concentration of one or more tryptophan gut metabolites selected from indoxyl sulfate, indole-3-propionic acid, 5-methoxytryptamine, indole-3-acetate, indole-3-lactate, or combinations thereof;
(c) determining baseline ratios of indoxyl sulfate concentration to the concentrations of indole-3-propionic acid, 5-methoxytryptamine, indole-3-acetate, and indole-3-lactate, wherein increased baseline ratios of indoxyl sulfate indicate that the subject is suffering from, or at risk of suffering from, depression, major depressive disorder, anxiety or combination thereof; and
(d) administering a therapy to the subject when increased ratios of indoxyl sulfate are determined.

24. The method of claim 23, wherein the subject is undergoing cognitive behavioral therapy, treatment with an anti-depressant, or a combination thereof.

25. The method of claim 24, wherein the anti-depressant comprises one or more of selective serotonin reuptake inhibitors (SSRI), tricyclic anti-depressants (TCA), selective serotonin and norepinephrine reuptake inhibitors (SNRI), monoamine oxidase inhibitors (MAOI), anxiolytics, antipsychotics, or combinations thereof.

26. The method of claim 25, wherein the anti-depressant comprises one or more of citalopram (Celexa®), escitalopram (Lexapro®), duloxetine (Cymbalta®), fluoxetine (Prozac®), paroxetine (Paxil®), sertraline (Zoloft®), trazodone (Desyrel®), lorazepam (Ativan®), oxazepam (Serax®), aripiprazole (Abilify®), clozapine (Clozaril®), haloperidol (Haldol®), olanzapine (Zyprexa®), quetiapine (Seroquel®), risperidone (Risperdal®), ziprasidone (Geodon®), amitriptyline, amoxapine, desipramine (Norpramin®), doxepin, imipramine (Tofranil®), nortriptyline (Pamelor®), protriptyline, trimipramine, or combinations thereof.

27. The method of claim 26, wherein the anti-depressant comprises escitalopram (Lexapro®), duloxetine (Cymbalta®), or a combination thereof.

28. The method of claim 23, wherein the measuring the concentration of one or more tryptophan gut metabolites is repeated at least twice, wherein a first measurement is taken as a baseline reading and a second or subsequent measurement is taken following administration of the therapy to the subject.

29. The method of claim 23, wherein the measured baseline concentrations and determined baseline ratios are compared to a healthy control.

30. The method of claim 23, wherein the therapy comprises one or more therapeutic agents to trap and sequester indoxyl sulfate.

31. The method of claim 30, wherein the therapeutic agent comprises AB-2004 or activated charcoal.

32. The method of claim 23, wherein the therapy comprises a probiotic gut bacterial strain that produces indole-3-propionic acid and does not produce indoxyl sulfate.

33. The method of claim 30, wherein the probiotic gut bacterial strain comprises Clostridium sporogenes.

34. The method of claim 23, wherein the therapy reduces tryptophanase expressing bacteria.

35. The method of claim 23, wherein the therapy comprises one or more therapeutic agents to inhibit enzymatic activity of sulfotransferase liver enzymes that produce indoxyl sulfate.

36. The method of claim 23, wherein the therapy comprises one or more therapeutic agents to inhibit aryl hydrocarbon receptor activity of intestinal immune cells.

37. The method of claim 23, further comprising performing functional magnetic resonance imaging of the subject prior to, during, or following administration of the therapy to the subject to monitor changes in brain resting state functional connectivity.

38. The method of claim 23, further comprising assessing disease severity of the subject prior to, during, and following administration of the therapy to the subject.

39. The method of claim 38, wherein assessing disease severity comprises the use of one or more disease assessment tests selected from a Quick Inventory of Depressive Symptomatology Self-Report (QIDS-SR), a Hamilton Anxiety Rating Scale (HAM-A), or a Hamilton Depression Rating Scale (HAM-D).

40. A method for stratifying one or more subjects suffering from a neuropsychiatric disease, disorder, or condition, including depression, major depressive disorder, anxiety, or combinations thereof into subgroups based on individual gut microbiome composition, enzymatic activity, concentrations and ratios of metabolites produced by gut bacteria, or combinations thereof, the method comprising:
(a) obtaining one or more biological samples from the subjects;
(b) measuring a baseline concentration of one or more tryptophan gut metabolites selected from indoxyl sulfate, indole-3-propionic acid, 5-methoxytryptamine, indole-3-acetate, indole-3-lactate, or combinations thereof;
(c) determining a baseline ratio of indoxyl sulfate concentration to concentrations of indole-3-propionic acid, 5-methoxytryptamine, indole-3-acetate, indole-3-lactate, or combinations thereof;
(d) stratifying the subjects into subgroups based on the determined baseline ratios of indoxyl sulfate concentration to concentrations of indole-3-propionic acid, 5-methoxytryptamine, indole-3-acetate, indole-3-lactate, or combinations thereof; and
(e) assessing disease severity of subjects in each stratified subgroup.

41. The method of claim 40, wherein increased ratios of indoxyl sulfate concentration to concentrations of indole-3-propionic acid, 5-methoxytryptamine, indole-3-acetate, and indole-3-lactate indicate that the subject is suffering from, or at risk of suffering from, depression, major depressive disorder, anxiety, or combinations thereof.

42. The method of claim 40, wherein the measured baseline concentrations and determined baseline ratios are compared to a healthy control.

43. The method of claim 40, wherein assessing disease severity of subjects in each stratified subgroup comprises using one or more disease assessment tests selected from a Quick Inventory of Depressive Symptomatology Self-Report (QIDS-SR), a Hamilton Anxiety Rating Scale (HAM-A), or a Hamilton Depression Rating Scale (HAM-D).

44. The method of claim 40, further comprising administering a therapy to subjects in each stratified subgroup based on the assessed disease severity and the determined baseline ratios of indoxyl sulfate concentration to concentrations of indole-3-propionic acid, 5-methoxytryptamine, indole-3-acetate, indole-3-lactate, or combinations thereof.

45. The method of claim 44, wherein the therapy comprises modulating the gut microbiome composition or enzymatic activity of the subject by:
(a) increasing the level of indole-3-propionic acid producing bacteria;
(b) decreasing the level of indoxyl sulfate producing bacteria;
(c) inhibiting bacterial enzymes that produce indoxyl sulfate;
(d) activating bacterial enzymes that produce indole-3-propionic acid; or
a combination of any of (a)-(d).

46. Use of a therapeutic agent to trap and sequester indoxyl sulfate for the treatment of a subject suffering from, or at risk of suffering from, depression, major depressive disorder, anxiety, or combinations thereof.

47. Use of an indole-3-propionic acid producing probiotic gut bacterial strain for the treatment of a subject suffering from, or at risk of suffering from, depression, major depressive disorder, anxiety, or combinations thereof.

48. The use of claim 47, wherein the probiotic gut bacterial strain comprises Clostridium sporogenes.

49. Use of a therapeutic agent to inhibit enzymatic activity of sulfotransferase liver enzymes that produce indoxyl sulfate for the treatment of a subject suffering from, or at risk of suffering from, depression, major depressive disorder, anxiety, or combinations thereof.

50. Use of a therapeutic agent to inhibit aryl hydrocarbon receptor activity of intestinal immune cells for the treatment of a subject suffering from, or at risk of suffering from, depression, major depressive disorder, anxiety, or combinations thereof.

51. A method for treating a subject suffering from, or at risk of suffering from, immune metabolic depression, anxiety, or combinations thereof by characterizing the gut microbiome metabolic profile of the subject, the method comprising:
(a) obtaining one or more biological samples from the subject;
(b) measuring a concentration of one or more tryptophan gut metabolites selected from indoxyl sulfate, indole-3-propionic acid, 5-methoxytryptamine, indole-3-acetate, indole-3-lactate, or combinations thereof;
(c) determining ratios of indoxyl sulfate concentration to the concentrations of indole-3-propionic acid, 5-methoxytryptamine, indole-3-acetate, indole-3-lactate, or combinations thereof, wherein increased ratios of indoxyl sulfate indicate that the subject is suffering from, or at risk of suffering from, depression, major depressive disorder, anxiety, or combinations thereof;
(d) measuring aryl hydrocarbon receptor activity of intestinal immune cells; and
(e) administering a therapy to the subject when increased ratios of indoxyl sulfate are determined.

52. The method of claim 51, wherein the measured concentrations and determined ratios are compared to a healthy control.

53. The method of claim 51, wherein the therapy comprises modulating the gut microbiome composition or enzymatic activity of the subject by:
(a) increasing the level of indole-3-propionic acid producing bacteria;
(b) decreasing the level of indoxyl sulfate producing bacteria;
(c) inhibiting bacterial enzymes that produce indoxyl sulfate;
(d) activating bacterial enzymes that produce indole-3-propionic acid; or
a combination of any of (a)-(d).

54. The method of claim 51, wherein the therapy comprises one or more therapeutic agents to inhibit aryl hydrocarbon receptor activity of intestinal immune cells.
